(19) United States
(12) Patent Application Publication
(10) Pub. No.: US 2005/0008665 A1
(43) Pub. Date: Jan. 13, 2005

(54) Cosmetic or dermatological active ingredient combination

(75) Inventors: Jan Batzer, Hamburg (DE); Werner Berens, Chevy Chase, MD (US); Thomas Blatt, Wedel (DE); Reza Keyhani, Hamburg (DE); Inge Kruse, Hamburg (DE); Claudia Mundt, Bremen (DE); Andreas Schepky, Hamburg (DE); Melanie Schmidt, Hamburg (DE); Uwe Schonrock, Nahe (DE); Jens Schulz, Schenefeld (DE); Christoph Smuda, Bonningstedt (DE); Franz Stab, Echem (DE); Rainer Wolber, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/824,102

(22) Filed: Apr. 13, 2004

Related U.S. Application Data
(63) Continuation of application No. PCT/EP02/11433, filed on Oct. 11, 2002.

(30) Foreign Application Priority Data
Oct. 13, 2001 (DE) 101 50 731.3
Oct. 13, 2001 (DE) 101 50 732.1
Oct. 13, 2001 (DE) 101 50 734.8
Oct. 13, 2001 (DE) 101 50 735.6
Oct. 13, 2001 (DE) 101 50 742.9
Dec. 22, 2001 (DE) 101 63 786.1

(51) Int. Cl. A61K 7/00

(52) U.S. Cl. 424/401

(57) ABSTRACT

The invention is a cosmetic or dermatological preparation comprising an active ingredient combination of at least one antioxidant or a derivative thereof and 8-hexadecene-1,16-dicarboxylic acid.

### CROSS-REFERENCE TO RELATED APPLICATIONS

This is a continuation application of PCT/EP02/11433, filed Oct. 11, 2002, which is incorporated herein by reference in its entirety, and also claims the benefit of German Priority Application Nos. 101 50 731.3, 101 50 732.1, 101 50 734.8, 101 50 735.6 and 101 50 742.9 filed Oct. 13, 2001; and German Priority Application No. 101 63 786.1, filed Dec. 22, 2001.

### FIELD OF THE INVENTION

The present invention relates to the use of active ingredients known per se for cosmetic and topical dermatological skin lightening or for preventing skin tanning, especially the skin tanning caused by UV radiation, and for lightening natural hair color.

In a preferred embodiment, the present invention relates to cosmetic and dermatological preparations for the prophylaxis and treatment of cosmetic or dermatological skin changes such as, for example, unwanted pigmentation, for example local hyperpigmentation and faulty pigmentation (for example moles, freckles), inhibition of natural pigmentation, but also for the purely cosmetic lightening of relatively large pigmented areas of skin which are perfectly appropriate for the individual skin type.

### BACKGROUND OF THE INVENTION

Melanocytes are responsible for pigmentation of the skin and are to be found in the lowest layer of the epidermis, the basal-cell layer, together with the basal cells as pigment-forming cells which occur—depending on the skin type either sporadically or else more or less frequently. Melanocytes contain melanosomes as characteristic cell organelles, in which the melanin is formed. There is increased formation of melanin inter alia on stimulation by UV radiation. This melanin is transported via the living layers of the epidermis (keratinocytes) finally into the horny layer (corneocytes) and causes a more or less pronounced brownish to brown-black skin color. Melanin is formed as the final stage of an oxidative process in which tyrosine is converted with involvement of the enzyme tyrosinase via several intermediate stages into the brown to brown-black eumelanins (DHICA- and DHI-melanin) and with involvement of sulfur-containing compounds into reddish pheomelanin. DHICA- and DHI-melanin are produced via the common intermediate stages of dopaquinone and dopachrome. The latter is converted, partly with the involvement of further enzymes, either into indole-5,6-quinonecarboxylic acid or into indole-5,6-quinone, from which the two eumelanins mentioned are produced. The production of pheomelanin proceeds inter alia via the intermediates dopaquinone and cysteinyidopa.

In a similar way to the pigmentation of the skin, melanin-producing melanocytes are also responsible for hair color (pigmentation of the hair). The amount and composition of the melanin in the hair determines the natural hair color, which is genetically fixed.

Problems with hyperpigmentation of the skin have diverse causes and are concomitant phenomena of many biological processes, e.g. UV radiation (e.g. freckles, ephilides), genetic disposition, faulty pigmentation of the skin during wound healing or scarring or skin aging (e.g. lentigines seniles).

Active ingredients and preparations counteracting skin pigmentation are known. The products in practical use are essentially based on hydroquinone, but, on the one hand, show their effect only after several weeks of use and, on the other hand, excessively long use thereof is objectionable for toxicological reasons. Inhibition of tyrosinase with substances such as kojic acid, ascorbic acid and azeleic acid and derivatives thereof is also common but has cosmetic and dermatological disadvantages.

The methods used to lighten hair color are mostly strongly prooxidative and may damage the hair severely. Well known in this connection is in particular the bleaching of hair with hydrogen peroxide.

### SUMMARY OF THE INVENTION

It was an object of the present invention to remedy these deficiencies.

8-Hexadecene-1,6-dicarboxylic acid (dioic acid, CAS number 20701-68-2; provisional INCI name octadecenedioic acid) is a metabolic product of yeast cells of the Candida strain. It is characterized by the following structure:

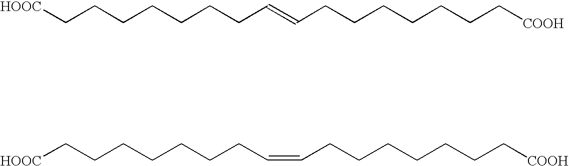

A fatty acid purely of plant origin serves as starting substance. This is converted into the hydroxy fatty acid which is then oxidized to the fatty aldehyde and finally to the dicarboxy acid. The yeast cells are derived from selected mutant strains. The commercial product has a purity of 95%. 8-Hexadecene-1,16-dicarboxylic acid is in this case in the form of a mixture of the cis and trans isomers, the amount of the cis isomer predominating. Oleic acid may also be present in the product in a concentration of approximately 3%. Among the solvents and oils, particularly suitable as solvents for dioic acid for cosmetic formulations are ethanol and ethanol/water mixtures (e.g. 50:50), dibutyl adipate, cetearyl ethylhexanoate, isopropyl myristate, ethyl hexylcocoate, C12-15 alkyl benzoate, cetearyl hexanoate, polypropylene glycol 3-myristyl ether, polypropylene glycol 15-stearyl ether, dicaprylyl carbonate, isohexadecane, octyidodecanol and 2-ethylhexyl methoxycinnamate, very particularly preferably octyidodecanol, isohexadecane, C12-15 alkyl benzoate and dibutyl adipate.

Antioxidants generally prevent oxidation processes. The effect of the antioxidant consists of terminating the autoxidative chain reaction or enhancing the antioxidant effect of antioxidants which are already present, regenerating their activity or inhibiting the activity of substances promoting autooxidation.

It is possible in principle at the cellular level to distinguish between enzymatic and non-enzymatic antioxidants, with the non-enzymatic antioxidants being divided into hydrophilic and lipophilic ones. The levels of the various antioxidants differ in individual tissues. Since the skin, depending on the point of view, is composed of at least 2 tissues, the epithelial epidermis and the connective-tissue dermis, a different distribution of the antioxidants can also be observed in the skin. It can generally be said that the epidermis, as the outermost barrier of the body towards the environment, is far better equipped with antioxidants than is the dermis. Enzymatic and non-enzymatic antioxidants are in part closely linked with one another and between one another via regenerative reaction pathways in which the energy metabolism of the cell plays an important part.

Since the mode of action of antioxidants intrinsic to the cell is lastly based on redox reactions, reducing equivalents are necessary for regeneration of the oxidized antioxidants. These reducing equivalents are provided in the form of NAD(P)H from the energy metabolism. In the epidermis, it appears that the oxidative part of the pentose phosphate pathway with glucose-6-phosphate dehydrogenase is particularly important in the synthesis of NADPH. Further sources of reducing equivalents besides this are the isocitrate shuttle and the malate shuttle with the enzymes isocitrate dehydrogenase and the malic enzyme, respectively.

Reduced glutathione (GSH) has particular importance because of its close linkage with other enzymatic (e.g. glutathione peroxidase) and non-enzymatic (viamin C, vitamin E) antioxidant systems and its regeneration directly via reducing equivalents from energy metabolism. GSH is the tripeptide γglutamyl-cysteyl glycine which has a sulfhydryl group as a functional group and which occurs in keratinocytes in a concentration of 1-2 mM and is thus the most frequently occurring free thiol inside cells. Its level in the epidermis is 2-5 times that in the dermis.

Besides its involvement in other antioxidant systems, GSH can itself also act as antioxidant. In this connection, GSH is particularly important in the reduction of oxidized sulfhydryl groups in proteins, because reduced sulfhydryl groups are frequently essential for the function of proteins. Besides this property, GSH can also react with free radicals and, like many other thiols too, with singlet oxygen and thus reduce oxidative stress.

Oxidized glutathione (GSSG), which results in the reactions mentioned, in the regeneration of other antioxidants and in the GSH-dependent enzymatic antioxidant reactions (see below), is reduced again by glutathione reductase with NADPH as cofactor. Glutathione reductase shows a distinctly higher activity in the epidermis, where its level is similar to that in the liver, than in the dermis.

Besides glutathione, the principal intracellular water-soluble antioxidant is ascorbate (vitamin C). It reacts likewise with singlet oxygen, superoxide, hydrogen peroxide and the highly reactive hydroxyl radical and is four times more common in the epidermis than in the dermis. In addition, ascorbate reacts with the free vitamin E radical and is thus involved in the regeneration of oxidized vitamin E to give the functional antioxidant (see below). Reaction of ascorbate with a free radical results in the semialdehyde ascorbate free radical which has only low reactivity and either undergoes non-enzymatic disproportionation through reaction with a further semialdehyde ascorbate free radical, resulting finally in ascorbate again, or is regenerated by NADH- and GSH-dependent ascorbate reductases to the antioxidant.

Uric acid is an antioxidant which acts primarily in the plasma, but is also found in the human skin. It occurs in the epidermis in a concentration five times higher than in the dermis.

Tocopherols (vitamin E), of which α-tocopherol is predominantly found in mammalian tissues as the most efficient free-radical scavenger, is the principal membrane-bound antioxidant. It suppresses the production of lipid free radicals and lipid peroxidation by interrupting the chain reaction described above. The level of tocopherols in the epidermis is about twice that in the dermis. As already described, the oxidized tocopherol free radical is reduced by ascorbate.

Ubiquinone and ubiquinol are encompassed by the term coenzyme Q, with ubiquinol being the more efficient antioxidant and coming close in activity to tocopherol. The epidermal content of ubiquinol-10, i.e. ubiquinol with 10 isoprene units, is 9 times higher than the dermal content in human skin.

As precursor of vitamin A, β-carotene is likewise important as antioxidant in the human skin. Its antioxidant effect is known as singlet oxygen quencher.

It can in principle be said that enzymatic antioxidant systems operate more efficiently than non-enzymatic antioxidants in intercepting oxidative stress.

The selenium-containing enzyme glutathione peroxidase occurs in the cytoplasm as a tetramer, metabolizes both hydrogen peroxide and organic peroxides and can be induced by oxidative stress. The enzyme is also found to a small extent within mitochondria. Besides the tetrameric selenoenzyme there is also a monomeric selenium-containing enzyme which is specific for phospholipid hydroperoxides. In relation to distribution in the skin, approximately equal ratios are found in the epidermis and dermis. The activity of the keratinocytes, which are closest to the surroundings of the body, is equipped with better defenses against oxidative stress because they are exposed most thereto.

Some GSH S-transferase subtypes, i.e. the alpha forms, likewise have organic peroxidase activity. It has to date been possible to detect in cultivated human keratinocytes only the pi form, whereas the alpha form is also found in human skin.

The glutathione peroxidases, i.e. the selenoenzymes and the GSH S-transferases with peroxidase activity, can be viewed in direct association with the other components of the GSH redox system. The functions of GSH, GSH reductase and the NAD(P)H-providing metabolic pathways in this regard have already been dealt with above.

Catalases, which degrade hydrogen peroxide via a dismutation reaction, have an activity in the epidermis which is four times that in the dermis.

Further important enzymes with antioxidant activity are the superoxide dismutatases (SOD). They catalyze the formation of hydrogen peroxide from superoxide anions and protons. The resulting hydrogen peroxide is metabolized by the mechanisms described previously. A distinction is made between the cytoplasmic Cu²⁺/Zn²⁺ and the mitochondrial Mn²⁺ isoforms, which occur in the skin in the ratio of 3:1. Overall, the SOD activity in the skin is 5-10 times higher than in other tissues, with scarcely any differences between epidermis and dermis. A response to oxidative stress is compensatory increase in SOD in the skin.

NAD(P)H:quinone reductase likewise belongs to the enzymatic antioxidants occurring in the skin. The enzyme is expressed in similar levels in human keratinocytes and in the liver and can moreover be induced by various xenobiotics and by oxidative stress.

Thioredoxin reductase has, besides its important function in DNA synthesis, also antioxidant properties in the skin and is found both in the cytoplasm and on the cell surface of keratinocytes. The thioredoxin reductase/thioredoxin system appears, besides its free radical-trapping properties, also to be involved in other antioxidant reactions.

The following antioxidants can be used in cosmetics: amino acids, imidazoles, peptides, carotenoids, carotenes, retinoids, α-lipoic acid, aurothioglucose, propylthiouracil and other thiols, dilauryl thiodipropionate, distearyl thiodipropionate, thiodipropionic acid and sulfoximine compounds, also chelators, α-hydroxy acids, humic acid, bile acid, bile extracts, bilirubin, biliverdin, EDTA, EGTA, unsaturated fatty acids, 2-aminopropionic acid diacetic acid, flavonoids, polyphenols, catechols, uniquinone and ubiquinol, vitamin C, tocopherols, and coniferyl benzoate of gum benzoin, rutinic acid, ferulic acid, butylhydroxytoluene, butylhydroxyanisole, nordihydroguaiaretic acid, nordihydroguaiaretic acid, trihydroxybutyrophenone, uric acid, mannose, zinc and selenium compounds, stilbenes.

However, none of this was able to smooth the way to the present invention.

It has therefore surprisingly emerged in a manner entirely unpredictable for the skilled worker that cosmetic or dermatological preparations comprising active ingredient combinations of at least one antioxidant or its derivative and 8-hexadecene-1,16-dicarboxylic acid remedy the disadvantages of the prior art.

It is preferred in this connection for the antioxidant or antioxidants to be selected from the group of imidazoles (e.g. urocanic acid), peptides, such as D,L-carnosine, D-carnosine, L-carnosine (e.g. anserine), carotenoids, such as, for example, α-carotene, β-carotene, lycopene and phytoene, α-lipoic acid (e.g. dihydrolipoic acid), lipoamide, aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, and sulfoximine compounds (e.g. buthionine sulfoximines, homocysteine sulfoximine, buthionine sulfones, penta-, hexa-, heptathionine sulfoximine) in very low tolerated doses (e.g. pmol to pmol/kg), metal chelators (e.g. α-hydroxy fatty acids, palmitic acid, phytic acid, lactoferrin, EDTA, EGTA, ferritin, desferal), humic acid, bile acid, bile extracts, bilirubin, biliverdin, unsaturated fatty acids (e.g. γ-linolenic acid, linoleic acid, oleic acid), folic acid, flavenoids (e.g. alpha glycosylrutin), polyphenols, catechols, melanins, ubiquinone and ubiquinol, vitamin C (e.g. ascorbyl palmitate, Mg ascorbyl phosphate, ascorbyl acetate), tocopherols (e.g. vitamin E acetate), rutinic acids, ferulic acids, butylhydroxytoluene, butylhydroxyanisole, nordihydroguaiaretic acid, trihydroxybutyrophenone, kojic acid, uric acid, mannoses, zinc and salts thereof (e.g. ZnO, ZnSO₄), selenium compounds (e.g. selenomethionine, erbselen) and/or nordihydroguaiaretic acid, enzymatic antioxidants such as, for example, catalase, superoxide dismutases, glutathione peroxidases, thioredoxin reductase and the corresponding enzyme mimetics.

It is particularly preferred for the antioxidant or antioxidants to be selected from the group of urocanic acid, phytoene, lipoid acid, lipoamide, ferritin, desferal, billirubin, billiverdin, melanins, ubiquinone, ubiquinol, vitamin C and its derivatives, ascorbyl palmitate, Mg ascorbyl phosphate, ascorbyl acetate, tocopherols and derivatives such as vitamin E acetate, uric acid, α-glucosylrutin, calalase and the superoxide dismutase.

It is very particularly preferred for the antioxidant or antioxidants to be selected from the group of urocanic acid, lipoic acid, lipoamide, melamins, ubiquinone, α-tocopherol, uric acid and catalase.

It is moreover preferred for the concentrations of antioxidant or antioxidants if vitamin E and/or derivatives thereof are not used as antioxidants to be 0.001 to 30% by weight, particularly preferably 0.05 to 20% by weight, in particular 0.1 to 10% by weight, if vitamin E and/or derivatives thereof are used as antioxidants to be 0.001 to 10% by weight and the concentration of 8-hexadecene-1,16-dicarboxylic acid to be 0.001 to 10% by weight, preferably 0.005 to 8% by weight, particularly preferably 0.05 to 5% by weight, in each case based on the total weight of the preparations.

The preparations are preferably used to counter unwanted pigmentation of the skin and/or for the treatment of pigmentation disorders and to counter unwanted pigmentation of the hair and/or for lightening the hair.

It is advantageous to use 8-hexadecene-1,16-dicarboxylic acid also in the form of one of its enantiomers.

### DETAILED DESCRIPTION OF THE PREFERRED EMBODIMENTS

Antioxidants in combination with 8-hexadecene-1,16-dicarboxylic acid, collectively also referred to as “active ingredient combination of the invention” or “active ingredient of the invention”, has proved to be excellently effective against unwanted pigmentation, in particular local hyperpigmentation and against the skin tanning caused by UV radiation, in particular both preventively and in the sense of a treatment. However, it is also extremely advantageous according to the invention to use the active ingredient used according to the invention or cosmetic or topical dermatological preparations with an effective content of active ingredient used according to the invention for the cosmetic or dermatological treatment of unwanted skin pigmentation, i.e. for example inhomogeneous pigmentation of aging skin, lentigines seniles or post-inflammatory hyperpigmentation.

α-Lipoic acid was isolated in 1952 from liver tissue and its structure was elucidated as the sulfur-containing fatty acid.

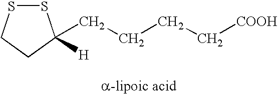

Bacteria, plants and higher organisms are able to produce α-lipoic acid themselves in their metabolism, but it is still an open question whether humans have their own biosynthesis.

α-Lipoic acid is employed for the therapy of polyneuropathy, an impairment of sensitivity of the hands and feet as a late consequence of diabetes. 200 to 600 milligrams of α-lipoic acid per day lead to a significant reduction in the intensity of pain. Energy metabolism of the nerves of the hands and feet is activated by α-lipoic acid, thus resulting in better nerve conductivity and therefore fewer feelings of numbness and losses of reflexes.

α-Lipoic acid reduces pathologically elevated liver function test results and promotes the cure of hepatitis. α-Lipoic acid is present in small amounts in most foodstuffs, but relatively high levels are to be found only in meat. It is acknowledged that α-lipoic acid has strongly antioxidant properties.

WO 97/10808 and U.S. Pat. No. 5,472,698 describe the cosmetic use of α-lipoic acid for symptoms of skin aging. DE-42 42 876 describes active ingredient combinations of biotin and antioxidants with α-lipoic acid for cosmetic and/or dermatological care of the skin and/or of the skin appendages, and cosmetic and/or dermatological preparations containing such active ingredient combinations.

It was therefore surprising and completely unpredictable for the skilled worker that cosmetic and/or dermatological preparations which comprise active ingredient combinations of α-lipoic acid and of plant and animal extracts containing the same with 8-hexadecene-1,16-dicarboxylic acid remedy the disadvantages of the prior art. It is moreover advantageous for α-lipoic acid or plant and animal extracts containing the same to be present in concentrations of 0.001-10% by weight preferably 0.01-5% by weight, in particular 0.1-2.0% by weight, and for 8-hexadecene-1,16-dicarboxylic acid to be present in a total concentration 0.001-10% by weight, preferably 0.005-8% by weight, in particular 0.05-5% by weight, in each case based on the total weight of the preparations. The invention also encompasses the use of such cosmetic and dermatological preparations for unwanted pigmentation of the skin and/or for the treatment of pigmentation disorders. Moreover, said active ingredient combination reduces unwanted pigmentation of the hair and thus brings about lightening of the hair.

It is advantageous also to use 8-hexadecene-1,16-dicarboxylic acid in the form of one of its enantiomers.

α-Lipoic acid in combination with 8-hexadecene-1,16-dicarboxylic acid, collectively also referred to as “active ingredient combination of the invention” or “active ingredient of the invention”, have proved to be excellently effective against unwanted pigmentation, in particular local hyperpigmentation and against the skin tanning caused by UV radiation, in particular both preventively and in the sense of a treatment. However, it is also extremely advantageous according to the invention to use the active ingredient used according to the invention or cosmetic or topical dermatological preparations with an effective content of active ingredient used according to the invention for the cosmetic or dermatological treatment of unwanted skin pigmentation, i.e. for example inhomogeneous pigmentation of aging skin, lentigines seniles or post-inflammatory hyperpigmentation.

Carnosine occurs in muscle tissue of humans and many animals; in birds it is replaced by the methyl derivative anserine. Little is known about the function of carnosine in the body; it appears to exercise a neurotransmitter function in the olfactory nerves.

The chemical structure of L-carnosine is characterized as follows:

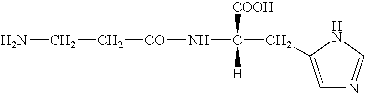

It is true than WO 94/9421245 describes the use of L-carnosine or combinations of the active ingredients L-carnosine and cis-urocanic acid or L-carnosine and trans-urocanic acid or L-carnosine and mixtures of cis- and trans-urocanic acids where appropriate in each case combined with one or more antioxidants and, where appropriate, with a suitable carrier for cosmetic and dermatological purposes, in particular for the prophylaxis and treatment of photosensitive skin, in particular photodermatoses and preferably polymorphic photodermatosis. Nevertheless, the prior art was unable to point towards the present invention.

It was therefore surprising and completely unpredictable for the skilled worker that cosmetic and/or dermatological preparations which comprise active ingredient combinations of carnosine and of plant and animal extracts containing the same with 8-hexadecene-1,16-dicarboxylic acid remedy the disadvantages of the prior art. It is preferred for these preparations to contain L-carnosine. It is moreover advantageous for carnosine or plant and animal extracts containing the same to be present in concentrations of 0.001-10% by weight, particularly preferably 0.01-1% by weight and for 8-hexadecene-1,16-dicarboxylic acid to be present in a total concentration 0.001-10% by weight, preferably 0.005-8% by weight, in particular 0.05-5% by weight, in each case based on the total weight of the preparations. The invention also encompasses the use of such cosmetic and dermatological preparations for unwanted pigmentation of the skin and/or for the treatment of pigmentation disorders. Moreover, said active ingredient combination reduces unwanted pigmentation of the hair and thus brings about lightening of the hair.

It is advantageous also to use 8-hexadecene-1,16-dicarboxylic acid in the form of one of its enantiomers.

Carnosine in combination with 8-hexadecene-1,16-dicarboxylic acid, collectively also referred to as “active ingredient combination of the invention” or “active ingredient of the invention”, has proved to be excellently effective against unwanted pigmentation, in particular local hyperpigmentation and against the skin tanning caused by UV radiation, in particular both preventively and in the sense of a treatment. However, it is also extremely advantageous according to the invention to use the active ingredient used according to the invention or cosmetic or topical dermatological preparations with an effective content of active ingredient used according to the invention for the cosmetic and dermatological treatment of unwanted skin pigmentation, i.e. for example inhomogeneous pigmentation of aging skin, lentigines seniles or post-inflammatory hyperpigmentation.

Ubiquinones are distinguished by the structural formula 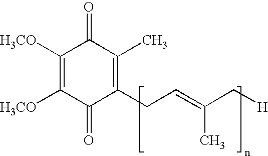
and represent the most widespread and thus best investigated bioquinones. Ubiquinones are referred to as Q-1, Q-2, Q-3 etc. depending on the number of isoprene units linked in the side chain, or as U-5, U-10, U-15 etc. according to the number of C atoms. They occur preferentially with particular chain lengths, for example with n=6 in some microorganisms and yeasts. Q10 predominates in most mammals, including humans.

Coenzyme Q-10 for example is characterized by the following structural formula:

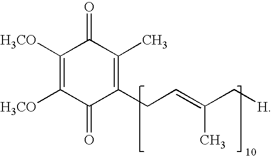

Ubiquinones act as electron carriers in the respiratory chain of organisms. They are located in the mitochondria, where they make the cyclic oxidation and reduction of the substrates of the citric acid cycle possible.

Plastoquinones have the general structural formula

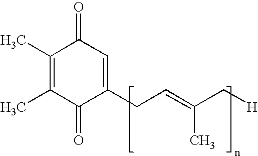

They can be isolated from chloroplasts and play a part as redox substrates in photosynthesis in the cyclic and noncyclic electron transport, being transformed reversibly into the corresponding hydroquinones (plastoquinol). Plastoquinones differ in the number n of isoprene residues and are designated correspondingly, e.g. PQ-9 (n=9). Other plastoquinones with different substituents on the quinone ring also exist.

Cosmetic preparations with coenzyme Q-10 are disclosed in DE-A-33 09 850 and are suitable for the treatment of skin disorders, for the prophylaxis of dystrophic and dysmetabolic states of the skin and for use in cases of chemical and physical respiratory damage or of sluggish respiration associated with age and debility.

Japanese published specification 58 180 410 describes the suitability of coenzyme Q-10 for cosmetics. It is said to activate the metabolism of skin cells and suppress oxidation. As a result, coenzyme Q10 has an important function in preventing skin damage from UV rays and preventing skin aging.

However, none of these documents was able to smooth the way to the present invention.

It was therefore surprising and could not have been predicted by the skilled worker that cosmetic and/or dermatological preparations which contain active ingredient combinations of bioquinones, and of plant and animal extracts comprising the same, with 8-hexadecene-1,16-dicarboxylic acid remedy the disadvantages of the prior art. It is preferred for these preparations to contain coenzyme Q10 as bioquinone. It is moreover advantageous for bioquinones or plant and animal extracts containing the same to be present in concentrations of 0.000001-5% by weight, particularly preferably 0.01-1% by weight and for 8-hexadecene-1,16-dicarboxylic acid to be present in a total concentration 0.001-10% by weight, preferably 0.005-8% by weight, in particular 0.05-5% by weight, in each case based on the total weight of the preparations. The invention also encompasses the use of such cosmetic or dermatological preparations to counter unwanted pigmentation of the skin and/or for the treatment of pigment disorders. In addition, said active ingredient combination reduces the unwanted pigmentation of the hair and thus brings about lightening of the hair.

It is advantageous also to use 8-hexadecene-1,16-dicarboxylic acid in the form of one of its enantiomers.

Bioquinones in combination with 8-hexadecene-1,16-dicarboxylic acid, collectively also referred to as “active ingredient combination of the invention” or “active ingredient of the invention”, has proved to be excellently effective against unwanted pigmentation, in particular local hyperpigmentation and against the skin tanning caused by UV radiation, in particular both preventively and in the sense of a treatment. However, it is also extremely advantageous according to the invention to use the active ingredient used according to the invention or cosmetic or topical dermatological preparations with an effective content of active ingredient used according to the invention for the cosmetic or dermatological treatment of unwanted skin pigmentation, i.e. for example inhomogeneous pigmentation of aging skin, lentigines seniles or post-inflammatory hyperpigmentation.

3-[4-Hydroxyphenylsulfate ester]-2-aminopropionic acid or 3-[3-hydroxyphenyl-sulfate ester]-2-aminopropionic acid (tyrosine O-sulfate ester) or corresponding sulfonic esters or phosphate esters or their derivatives, and their plant and animal extracts and their esters are derived from the basic structure of tyrosine and are characterized by the following structures:

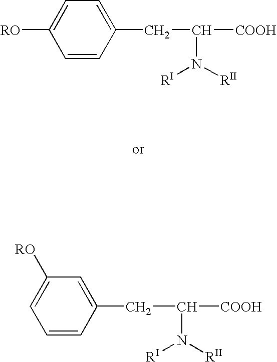

In these, R is a sulfate, phosphate or sulfonate or a derivative thereof, R′ and R″ are each a hydrogen or an alkyl group.

The preparation of 3-[4-hydroxyphenylsulfate ester]-2-aminopropionic acid or 3-[3-hydroxyphenylsulfate ester]-2-aminopropionic acid (tyrosine O-sulfate ester) or corresponding sulfonic esters or phosphate esters or their derivatives and their plant and animal extracts is described in the specialist literature.

DE 19720339 describes the use of 3-[4-hydroxyphenylsulfate ester]-2-aminopropionic acid or 3-[3-hydroxyphenylsulfate ester]-2-aminopropionic acid (tyrosine O-sulfate ester) or derivatives thereof as active ingredient in preparations for skin lightening. By contrast, the publications mentioned disclose nothing about the advantageous synergistic effect of the combination 3-[4-hydroxyphenylsulfate ester]-2-aminopropionic acid or 3-[3-hydroxyphenylsulfate ester]-2-aminopropionic acid with 8-hexadecene-1,16-dicarboxylic acid.

It was therefore surprising and could not have been predicted by the skilled worker that cosmetic or dermatological preparations comprising active ingredient combinations selected from the group of 3-[4-hydroxyphenylsulfate ester]-2-aminopropionic acid or 3-[3-hydroxyphenylsulfate ester]-2-aminopropionic acid (tyrosine O-sulfate ester) or corresponding sulfonic esters or phosphate esters or their derivatives and plant and/or animal extracts containing these substances in combination with 8-hexadecene-1,16-dicarboxylic acid remedy the disadvantages of the prior art. It is preferred in this connection for L-3-[4-hydroxyphenylsulfate ester]-2-aminopropionic acid or L-3-[3-hydroxyphenylsulfate ester]-2-aminopropionic acid (tyrosine O-sulfate ester) or corresponding sulfonic esters or phosphate esters or their derivatives and plant and/or animal extracts containing these substances, and 8-hexadecene-1,16-dicarboxylic acid to be present in an effective content in the preparations. It is particularly preferred to use L-3-[4-hydroxyphenylsulfate ester]-2-aminopropionic acid (tyrosine O-sulfate ester). It is very particularly preferred in this connection for the content of hydroxyphenylsulfate ester-2-aminopropionic acid or corresponding sulfonic esters or phosphate esters or their derivatives to be 0.001 to 10% by weight, preferably 0.01 to 5% by weight, in particular 0.1 to 2.0% by weight, based on the total weight of the preparations, and for 8-hexadecene-1,16-dicarboxylic acid to be present in a total concentration 0.001-10% by weight, preferably 0.005-8% by weight, in particular 0.05-5% by weight, in each case based on the total weight of the preparations.

Such preparations can be used advantageously to counter unwanted pigmentation of the skin, for the treatment of pigmentation disorders of the skin and/or for the treatment of age spots. In addition, said active ingredient combination reduces the unwanted pigmentation of the hair and thus causes lightening of the hair.

It is advantageous also to use 8-hexadecene-1,16-dicarboxylic acid in the form of one of its enantiomers.

3-[4-Hydroxyphenylsulfate ester]-2-aminopropionic acid or 3-[3-hydroxyphenyl-sulfate ester]-2-aminopropionic acid (tyrosine O-sulfate ester) in combination with 8-hexadecene-1,16-dicarboxylic acid, collectively also referred to as “active ingredient combination of the invention” or “active ingredient of the invention”, has proved to be excellently effective against unwanted pigmentation, in particular local hyperpigmentation and against the skin tanning caused by UV radiation, in particular both preventively and in the sense of a treatment. However, it is also extremely advantageous according to the invention to use the active ingredient used according to the invention or cosmetic or topical dermatological preparations with an effective content of active ingredient used according to the invention for the cosmetic or dermatological treatment of unwanted skin pigmentation, i.e. for example inhomogeneous pigmentation of aging skin, lentigines seniles or post-inflammatory hyperpigmentation.

Prophylaxis or cosmetic or dermatological treatment with the active ingredient used according to the invention or with the cosmetic or topical dermatological preparations having an effective content of active ingredient used according to the invention takes place in the conventional way, in particular by applying the active ingredient combination of the invention or the cosmetic or topical dermatological preparations with an effective content of active ingredient used according to the invention to the affected areas of skin. It has surprisingly emerged that the active ingredient combination of the invention fulfills the objects on which the invention is based. The active ingredient combinations of the invention act in all these uses synergistically in relation to the individual components.

It is advantageously possible for the active ingredient combination of the invention to be incorporated into conventional cosmetic and dermatological preparations which may be in various forms. Thus, for example, they may be a solution, an emulsion of the water-in-oil (W/O) type or of the oil-in-water (O/W) type, or a multiple emulsions, for example of the water-in-oil-in-water (W/O/W) or oil-in-water-in-oil (O/W/O) type, a hydrodispersion or lipodispersion, a gel, a solid stick, a transdermal therapeutic system or else an aerosol.

Emulsions of the invention for the purposes of the present invention, e.g. in the form of a cream, of a lotion, of a cosmetic milk are advantageous and comprise, for example, fats, oils, waxes and/or other lipids, and water and one or more emulsifiers normally used for such a formulation type.

It is also possible and advantageous for the purposes of the present invention to include the active ingredient used according to the invention in aqueous systems or surfactant preparations for the cleaning and care of the skin and hair. This includes both shower gels, shampoos but also conditioners, hair cures, hair rinses, hair tonics, sprays etc.

The skilled worker is, of course, aware that high-quality cosmetic compositions are in most cases inconceivable without the use of excipients and additives. These include, for example, bodying agents, fillers, perfume, dyes, emulsifiers, additional active ingredients such as vitamins or proteins, light protection agents, stabilizers, insect repellents, alcohol, water, salts, or substances having anti-microbial, proteolytic or keratolytic activity etc.

It is likewise advantageous to use the active ingredient 8-hexadecene-1,16-dicarboxylic acid in the form of molecular adducts with cyclodextrins. It is assumed that the cyclodextrin structures in this case act as host molecule and 8-hexadecene-1,16-dicarboxylic acid acts as guest molecule. For the preparation, cyclodextrins are dissolved in water, and 8-hexadecene-1,16-dicarboxylic acid is added. The molecular adduct then precipitates as solid and can be subjected to the usual purification and workup steps. It is known that cyclodextrin-guest complexes in an appropriate solvent (e.g. water) are in an equilibrium between the specific guest-cyclodextrin complex and the dissociated form, with cyclodextrin and guest possibly being separated to a certain extent. Such equilibrium systems are likewise advantageous for the purposes of the present invention.

Corresponding requirements apply mutatis mutandis to the formulation of medicinal preparations.

Medicinal topical compositions for the purposes of the present invention usually comprise one or more medicaments in effective concentration. For the sake of simplicity, reference is made for the clear distinction between cosmetic and medical applications and corresponding products to the statutory provisions of the Federal Republic of Germany (e.g. Cosmetic Regulations, Food and Drugs Act).

It is likewise advantageous in this connection for the active ingredient used according to the invention to be put as additive in preparations which already comprise other active ingredients for other purposes.

Correspondingly, cosmetic or topical dermatological compositions can be used for the purposes of the present invention, depending on their constitution, for example as skin-protecting cream, cleansing milk, sunscreen lotion, nutrient cream, day or night cream etc. It is possible and advantageous where appropriate to use the compositions of the invention as basis for pharmaceutical formulations.

It is also advantageous for the purposes of the present invention to produce cosmetic and dermatological preparations whose main purpose is not protection from sunlight but nevertheless comprise a content of further UV-protective substances. Thus, for example, UV-A and UV-B filter substances are normally incorporated into day creams or makeup products. UV-protective substances, just like antioxidants and, if desired, preservatives also represent an effective protection of the preparations themselves from spoilage. Also beneficial are cosmetic and dermatological preparations in the form of a sunscreen composition.

Accordingly, the preparations comprise for the purposes of the present invention preferably at least one further UV-A, UV-B and/or broad-spectrum filter substance. The formulations may, although not necessarily, where appropriate also comprise one or more organic and/or inorganic pigments as UV filter substances, which may be present in the aqueous and/or the oil phase.

The preparations of the invention may also advantageously be in the form of so-called oil-free cosmetic or dermatological emulsions which comprise an aqueous phase and at least one UV filter substance, which is liquid at room temperature, as further phase.

Particularly advantageous UV filter substances which are liquid at room temperature for the purposes of the present invention are homomenthyl salicylate (INCI: homosalate), 2-ethylhexyl 2-cyano-3,3-diphenylacrylate (INCI: octocrylene), 2-ethylhexyl 2-hydroxybenzoate (2-ethylhexyl salicylate), octyl salicylate, INCI: octyl salicylate) and esters of cinnamic acid, preferably 4-methoxycinnamic acid (2-ethylhexyl)ester (2-ethylhexyl 4-methoxycinnamate, INCI: octyl methoxycinnamate) and 4-methoxycinnamic acid isopentyl ester (isopentyl 4-methoxycinnamate, INCI: isoamyl p-methoxycinnamate).

Preferred inorganic pigments are metal oxides and/or other metal compounds which are slightly soluble or insoluble in water, in particular oxides of titanium (TiO₂), zinc (ZnO), iron (e.g Fe₂O₃), zirconium (ZrO₂), silicon (SiO₂), manganese (e.g. MnO), aluminum (Al₂O₃), cerium (e.g. Ce₂O₃), mixed oxides of the corresponding metals, and blends of such oxides, and the sulfate of barium (BaSO₄).

The pigments may advantageously be used for the purposes of the present invention also in the form of commercially available oily or aqueous predispersions. It is possible and advantageous to add dispersion aids and/or solubilizers to these predispersions.

The pigments may according to the invention advantageously by surface-treated (“coated”), in which case the intention is for example to produce or maintain a hydrophilic, amphiphilic or hydrophobic character. This surface treatment may consist of providing the pigments by processes known per se with a thin hydrophilic and/or hydrophobic inorganic and/or organic layer. The various surface coatings may also for the purposes of the present invention contain water.

Inorganic surface coatings for the purposes of the present invention may consist of aluminum oxide (Al₂O₃), aluminum hydroxide Al(OH)₃, or aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also called silica, CAS No.: 7631-86-9), or iron oxide (Fe₂O₃). These inorganic surface coatings may occur alone, in combination and/or in combination with organic coating materials.

Organic surface coatings for the purposes of the present invention may consist of vegetable or animal aluminum stearate, vegetable or animal stearic acid, lauric acid, dimethylpolysiloxane (also: dimethicones), methylpolysiloxane (methicones), simethicones (a mixture of dimethylpolysiloxane with an average chain length of 200 to 350 dimethylsiloxane units and silica gel) or alginic acid. These organic surface coatings may occur alone, in combination and/or in combination with inorganic coating materials.

Zinc oxide particles and predispersions of zinc oxide particles suitable according to the invention are obtainable under the following proprietary names from the stated companies:

| Proprietary name | Coating | Manufacturer |
|---|---|---|
| Z-Cote HP1 | 2% dimethicone | BASF |
| Z-Cote | / | BASF |
| ZnO NDM | 5% dimethicone | H&R |

Suitable titanium dioxide particles and predispersions of titanium dioxide particles are obtainable under the following proprietary names from the stated companies:

| Proprietary name | Coating | Manufacturer |
|---|---|---|
| MT-100TV | aluminum hydroxide/stearic acid | Tayca Corporation |
| MT-100Z | aluminum hydroxide/stearic acid | Tayca Corporation |
| Eusolex T-2000 | alumina/simethicones | Merck KgaA |
| Titanium dioxide | Octyltrimethylsilane | Degussa |
| T805 | | |
| (Uvinul TiO₂) | | |

Advantageous UV-A filter substances for the purposes of the present invention are dibenzoylmethane derivatives, in particular 4-(tert-butyl)-4′-methoxydibenzoyl-methane (CAS No.: 70356-09-1), which is sold by Givaudan under the name Parsol® 1789 and by Merck under the proprietary name Eusolex® 9020.

Advantageous further UV filter substances for the purposes of the present invention are sulfonated, water-soluble UV filters such as, for example:
phenylene-1,4-bis(2-benzimidazyl)-3,3′-5,5′-tetrasulfonic acid and its salts, in particular the corresponding sodium, potassium or triethanolammonium salts, especially phenylene-1,4-bis(2-benzimidazyl)-3,3′-5,5′-tetrasulfonic acid bissodium salt with INCI name bisimidazylate (CAS No.: 180898-37-7), which is obtainable for example under the proprietary name Neo Heliopan AP from Haarmann & Reimer;
salts of 2-phenylbenzimidazol-5-sulfonic acid, such as its sodium, potassium or its triethanolammonium salt, and the sulfonic acid itself with the INCI name phenylbenzimidazole sulfonic acid (CAS No.: 27503-81-7), which is obtainable for example under the proprietary name Eusolex 232 from Merck or under Neo Heliopan Hydro from Haarmann & Reimer;
1,4-di(2-oxo-10-sulfo-3-bornylidenemethyl)benzene (also: 3,3′-(1,4-phenylene-dimethylene)bis(7,7-dimethyl-2-oxobicyclo[2.2.1]hept-1-ylmethanesulfonic acid) and its salts (in particular the corresponding 10-sulfato compounds, especially the corresponding sodium, potassium or triethanolammonium salt, which is also called benzene-1,4-di(2-oxo-3-bornylidenemethyl-10-sulfonic acid). Benzene-1,4-di(2-oxo-3-bornylidinemethyl-10-sulfonic acid) has the INCI name terephtalidene dicamphor sulfonic acid (CAS No.: 90457-82-2) and is obtainable for example under the proprietary name Mexoryl SX from Chimex; and
sulfonic acid derivatives of 3-benzylidenecamphor such as, for example, 4-(2-oxo-3-bornylidenemethyl)benzenesulfonic acid, 2-methyl-5-(2-oxo-3-bornylidene-methyl)sulfonic acid and salts thereof.

Advantageous UV filter substances for the purposes of the present invention are moreover so-called broad-spectrum filters, i.e. filter substances which absorb both UV-A and UV-B radiation.

Advantageous broad-spectrum filters or UV-B filter substances are, for example, triazine derivatives such as, for example,
2,4-bis{[4-(2-ethylhexyloxy)-2-hydroxy]phenyl}-6-(4-methoxyphenyl)-1,3,5-triazine (INCI: aniso triazine) which is obtainable under the proprietary name Tinosorb® S from CIBA-Chemikalien GmbH;
diethylhexylbutylamidotriazone (INCI: diethylhexylbutamidotriazone) which is obtainable under the proprietary name UVASORB HEB from Sigma 3V; and
4,4′,4″-(1,3,5-triazine-2,4,6-triyltriimino)tribenzoic acid tris(2-ethylhexyl ester), also: 2,4,6-tris[anilino-(p-carbo-2′-ethyl-1′-hexyloxy)]-1,3,5-triazine (INCI: ethylhexyl triazone) which is marketed by BASF Aktiengesellschaft under the commerical name UVINUL® T 150.

An advantageous broad-spectrum filter for the purposes of the present invention is also 2,2′-methylenebis(6-(2H-benzotriazol-2-yl)-4-(1,1,3,3-tetramethylbutyl)phenol) which is obtainable under the proprietary name Tinosorb® M from CIBA-Chemikalien GmbH.

A further advantageous broad-spectrum filter for the purposes of the present invention is 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name drometrizole trisiloxane, which is obtainable under the proprietary name Mexoryl® XL from Chimex.

The further UV filter substances may be oil-soluble or water-soluble.

Advantageous oil-soluble UV-B and/or broad-spectrum filter substances for the purposes of the present invention are, for example:
3-benzylidenecamphor derivatives, preferably 3-(4-methylbenzylidene)camphor, 3-benzylidenecamphor;
4-aminobenzoic acid derivatives, preferably 4-(dimethylamino)benzoic acid 2-ethylhexyl ester, 4-(dimethylamino)benzoic acid amyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzo-phenone
and UV filters bound to polymers.
3-(4-(2,2-bisethoxycarbonylvinyl)phenoxy)propenyl)methoxysiloxane/dimethyl-siloxane copolymer, which is obtainable for example under the proprietary name Parsol® SLX from Hoffmann La Roche.

Examples of advantageous water-soluble filter substances are sulfonic acid derivatives of 3-benzylidenecamphor such as, for example, 4-(2-oxo-3-bornylidenemethyl)benzenesulfonic acid, 2-methyl-5-(2-oxo-3-bornylidenemethyl)-sulfonic acid and salts thereof.

A further light protection filter substance to be used advantageously according to the invention is ethylhexyl 2-cyano-3,3-diphenylacrylate (octocrylene) which is obtainable from BASF under the name Uvinul® N 539.

Particularly advantageous preparations for the purposes of the present invention which are distinguished by a high or very high UV-A and/or UV-B protection comprise, besides the filter substance(s) of the invention, preferably also further UV-A and/or broad-spectrum filters, in particular dibenzoylmethane derivatives [for example 4-(tert-butyl)₄′-methoxydibenzoylmethane], phenylene-1,4-bis-(2-benzimidazyl)-3,3′-5,5′-tetrasulfonic acid and/or its salts, 1,4-di(2-oxo-10-sulfo-3-bornylidenemethyl)benzene and/or its salts and/or 2,4-bis{[4-(2-ethylhexyloxy)-2-hydroxy]phenyl}-6-(4-methoxyphenyl)-1,3,5-triazine, in each case singly or in any combinations with one another.

The list of UV filters mentioned as employable for the purposes of the present invention is, of course, not intended to be limiting.

The preparations of the invention advantageously contain the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the hair or the skin from the complete range of ultraviolet radiation.

The cosmetic and dermatological preparations of the invention may comprise cosmetic active ingredients, excipients and/or additives as are normally used in such preparations, e.g. antioxidants, preservatives, bactericides, perfumes, substances to prevent foaming, dyes, pigments having a coloring effect, thickeners, surface-active substances, emulsifiers, emollient, moisturizing and/or humectant substances, fats, oils, waxes or other conventional ingredients of a cosmetic or dermatological formulation such as alcohols, polyols, polymers, foam stabilizers, electrolytes, organic solvents or silicone derivatives.

Where the cosmetic or dermatological preparation for the purposes of the present invention is a solution or emulsion or dispersion, solvents which can be used are:
water or aqueous solutions
oils such as triglycerides of capric or caprylic acid, but preferably castor oil;
fats, waxes and other natural and synthetic fatty substances, preferably esters of fatty acids with alcohols of low C number, e.g. with isopropanol, propylene glycol or glycerol, or esters of fatty alcohols with alkanoic acids of low C number or with fatty acids; and
alcohols, diols or polyols of low C number, and the ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

Mixtures of the aforementioned solvents are used in particular. Water may be a further ingredient in the case of alcoholic solvents.

The oily phase of the emulsions, oleogels or hydrodispersions or lipodispersions for the purposes of the present invention is preferably chosen from the group of esters of saturated and/or unsaturated, branched and/or unbranched alkanecarboxylic acids with a chain length of 3 to 30 C atoms and saturated and/or unsaturated, branched and/or unbranched alcohols with a chain length of 3 to 30 C atoms. Ester oils of these types can then be chosen advantageously from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of such esters, e.g. jojoba oil.

The oily phase can also advantageously be chosen from the group of branched and unbranched hydrocarbons and hydrocarbon waxes, of silicone oils, of dialkyl ethers, of the group of saturated or unsaturated, branched or unbranched alcohols, and of fatty acid triglycerides, specifically the triglyceryl esters of saturated and/or unsaturated, branched and/or unbranched alkanecarboxylic acids with a chain length of 8 to 24, in particular 12-18 C atoms. The fatty acid triglycerides can, for example, advantageously be chosen from the group of synthetic, semisynthetic and natural oils, e.g. olive oil, sunflower oil, soybean oil, peanut oil, rapeseed oil, almond oil, palm oil, coconut oil, palm kernel oil and more of the like.

It is also advantageous to employ for the purposes of the present invention any mixtures of such oil and wax components. It may also be advantageous where appropriate to employ waxes, for example cetyl palmitate, as sole lipid component of the oily phase.

The oily phase is advantageously chosen from the group of 2-ethylhexyl isostearate, octyldodecanol, isotridecyl isononanoate, isoeicosane, 2-ethylhexyl cocoate, C₁₂₋₁₅-alkyl benzoate, carylic/capric acid triglyceride, dicapryl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Hydrocarbons which can be used advantageously for the purposes of the present invention are liquid paraffin, squalane and squalene.

The oily phase may advantageously also have a content of cyclic or linear silicone oils or consist completely of such oils, although it is preferred to use an additional content of other oily phase components apart from the silicone oil or the silicone oils.

It is advantageous to employ cyclomethicone (octamethylcyclotetrasiloxane) as silicone oil to be used according to the invention. However, other silicone oils can also be used advantageously for the purposes of the present invention, for example hexamethylcyclotrisiloxane, polydimethylsiloxane, poly(methylphenylsiloxane).

Also particularly advantageous are mixtures of cyclomethicone and isotridecyl isononanoate, of cyclomethicone and 2-ethylhexyl isostearate.

Gels used according to the invention normally contain alcohols of low C number, e.g. ethanol, isopropanol, 1,2-propanediol, glycerol and water or an aforementioned oil in the presence of a thickener which is preferably silicon dioxide or an aluminum silicate in the case of oily/alcoholic gels, and is preferably a polyacrylate in the case of hydroalcoholic or alcoholic gels.

Solid sticks comprise, for example, natural or synthetic waxes, fatty alcohols or fatty esters. Preference is given to lipcare sticks and stick formulations for body deodorization.

Conventional bases suitable for use as cosmetic sticks for the purposes of the present invention are liquid oils (e.g. liquid paraffin, castor oil, isopropyl myristate), semisolid ingredients (e.g. petrolatum, lanolin), solid ingredients (e.g. beeswax, ceresin and microcrystalline waxes or ozokerite) and high-melting waxes (e.g. carnauba wax, candelilla wax).

Suitable propellants for cosmetic and/or dermatological preparations which can be sprayed from aerosol containers for the purposes of the present invention are the usual known volatile, liquefied propellants, for example hydrocarbons (propane, butane, isobutane), which can be employed alone or mixed with one another. Compressed air can also be used advantageously.

The skilled worker of course knows that there are propellant gases which are non-toxic per se and which would in principle be suitable for implementing the present invention in the form of aerosol products but which ought not to be used nevertheless because of the objectionable effect on the environment or other accompanying circumstances, in particular hydrofluorocarbons and hydrochlorofluorocarbons (HCFC).

Cosmetic preparations for the purposes of the present invention may also be in the form of gels which, besides an effective content of active ingredient of the invention and solvents normally used therefor, preferably water, also contain organic thickeners, e.g. gum arabic, xanthan gum, sodium alginate, celllulose derivatives, preferably methylcellulose, hydroxymethylcellulose, hydroxyethylcellulose, hydroxypropylcellulose, hydroxypropylmethylcellulose or inorganic thickeners, e.g. aluminum silicates such as, for example, bentonites, or a mixture of polyethylene glycol and polyethylene glycol stearate or distearate. The thickener is present in the gel for example in an amount between 0.1 and 30% by weight, preferably between 0.5 and 15% by weight.

Preparations of the invention which are hair cosmetic cleansing preparations for the hair or the scalp may be in solid or liquid form. They preferably contain at least one anionic, nonionic or amphoteric surface-active substance or mixtures thereof, where appropriate an electrolyte and auxiliaries as are normally used for this purpose. The surface-active substance can be present in a concentration between 1 and 94% by weight in the cleansing preparations, based on the total weight of the preparations, but in particular between 1 and 50%—by weight.

It is possible in particular for aqueous cosmetic cleansing compositions of the invention or low-water or water-free cleansing composition concentrates intended for aqueous cleansing to comprise anionic, nonionic and/or amphoteric surfactants, for example to comprise conventional soaps, e.g. fatty acid salts of sodium, alkyl sulfates, alkyl ether sulfates, alkane- and alkylbenzene sulfonates, sulfoacetates, sulfobetaines, sarcosinates, amidosulfobetaines, sulfosuccinates, sulfosuccinic acid hemiesters, alkyl ether carboxylates, protein/fatty acid condensates, alkylbetaines and amidobetaines, fatty acid alkanolamides, polyglycol ether derivatives.

Anionic surfactants are preferably employed in concentrations between 5% by weight and 20% by weight. Suitable examples are sodium laureth sulfate as available under the name Texapon N 70 from the Henkel company, or disodium laureth sulfosuccinate as available under the name Rewopol SBFA 30 from the Witco company. Nonionic surfactants are preferably employed in concentrations of 1% by weight to 10% by weight. Examples are decyl glucoside as available under the name Oramix NS 10 from the Seppic company or polysorbate 80 as is available under the name Tween 80 from the ICI company. Amphoteric surfactants are preferably employed in concentrations of 1% by weight to 10% by weight. Examples are cocamidopropylbetaine as available as Tego Betaine from the Goldschmidt company, or sodium cocoamphoacetate as available under the name Miranol Ultra from the Rhone-Poulenc company.

The percentage data are based on the total weight of the preparations.

The hair cosmetic cleansing compositions may additionally comprise conditioning aids, e.g. in amounts of 0.001 to 10% by weight based on the total weight of the preparations. Preferred conditioning aids include polymeric quaternary compounds (quats). Polymeric quats are frequently employed in shampoos e.g. with a concentration of 0.01 to 2% by weight. They include polyquaternium-10 as available under the name Polymer JR 400 from the Amerchol company, or hydroxypropyl guar hydroxypropyltrimonium chloride as available under the name Jaguar C 162 from the Rhone-Poulenc company.

The preparations of the invention may comprise cosmetic excipients as are normally used in such preparations, e.g. preservatives, perfumes, substances to prevent foaming, foam stabilizers, dyes, pigments which have a coloring effect, thickeners, surface-active substances, emulsifiers, emollient, moisturizing and/or humectant substances, refatting agents, fats, oils, waxes, alcohols, polyols and their toxicologically acceptable ethers and esters, branched and/or unbranched hydrocarbons, further antioxidants, stabilizers, pH regulators, bodying agents, bactericides, deodorants, antimicrobial substances, antistatics, UV absorbers, complexing and sequestering agents, pearlescent agents, polymers, electrolytes, organic solvents, silicone derivatives, plant extracts, vitamins and/or other active ingredients or other usual ingredients of a cosmetic or dermatological formulation. Solubilizers may also be present for example for incorporating hydrophobic components such as, for example, perfume preparations.

The total amount of the excipients is, for example, 0.001 to 15% by weight, preferably 0.01 to 10% by weight, in each case based on the total weight of the preparation.

The water content of the preparations is, for example, 50 to 95% by weight, preferably 55 to 90% by weight, in each case based on the total weight of the preparation.

The pH of the preparations can be adjusted in a known manner by adding acids or bases, preferably by adding buffer mixtures, e.g. based on citric acid/citrate or phosphoric acid phosphate buffer mixtures. The pH is preferably below 10, e.g. in the range 4-8, in particular in the range 5-7.

The following examples are intended to illustrate the present invention.

All quantitative data, percentage data or parts are based, unless indicated otherwise, on the total weight of the preparations or the particular mixtures.

### EXAMPLES

1. PIT emulsions with antioxidants

| | 1 | 2 | 3 | 4 | 5 |
|---|---|---|---|---|---|
| Glycerol monosterate, self-emulsifying | 0.50 | | 3.00 | 2.00 | 4.00 |
| Polyoxyethylene 12 cetylstearyl ether | | 5.00 | | 1.00 | 1.50 |
| Polyoxyethylene 20 cetylstearyl ether | | | | 2.00 | |
| Polyoxyethylene 30 cetylstearyl ether | 5.00 | | 1.00 | | |
| Stearyl alcohol | | | 3.00 | | 0.50 |
| Cetyl alcohol | 2.50 | 1.00 | | 1.50 | |
| 2-Ethylhexyl methoxycinnamate | | | | 5.00 | 8.00 |
| 2,4-bis(4-(2-Ethylhexyloxy)-2-hydroxyl)- | | 1.50 | | 2.00 | 2.50 |
| phenyl)-6-(4-methoxyphenyl)-(1,3,5)- | | | | | |
| triazine | | | | | |
| 1-(4-tert-butylphenyl)-3-(4- | | | 2.00 | | |
| methoxyphenyl)-1,3-propanedione | | | | | |
| Diethylhexyl butamidotriazone | 1.00 | 2.00 | | 2.00 | |
| Ethylhexyltriazone | 4.00 | | 3.00 | 4.00 | |
| 4-Methylbenzylidenecamphor | | 4.00 | | | 2.00 |
| Octocrylene | | 4.00 | | | 2.50 |
| Phenylene-1,4-bis(monosodium, | | | 0.50 | | 1.50 |
| 2-benzimidazyl-5,7-disulfonic acid | | | | | |
| Phenylbenzimidazole sulfonic acid | 0.50 | | | 3.00 | |
| C12-C15 alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethylpolysiloxane | 2.00 | | | 0.50 | 0.50 |
| Shea butter | | 2.00 | | | 0.50 |
| PVP hexadecene copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| Dioic acid | 0.15 | 1.00 | 0.20 | 0.40 | 0.10 |
| Uric acid | 0.75 | 0.2 | 0.50 | 0.20 | 0.40 |
| Alpha-glycosylrutin | 0.10 | | 0.20 | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

2. O/W cream with antioxidants

| Examples | 1 | 2 | 3 | 4 | 5 |
|---|---|---|---|---|---|
| Glyceryl stearate citrate | | | 2.00 | | 2.00 |
| Glyceryl sterate, self-emulsifying | 4.00 | 3.00 | | | |
| PEG 40 stearate | 1.00 | | | | |
| Polyglyceryl 3-methyl-glucose distearate | | | | 3.00 | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetylstearyl alcohol | | | | | 2.00 |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Liquid paraffin | 5.00 | 2.00 | | 3.00 | |
| Titanium dioxide | | | 1.00 | | |
| 4-Methylbenzylidenecamphor | | | 1.00 | | |
| 1-(4-tert-Butylphenyl)-3-(4-methoxy- | | | 0.50 | | |
| phenyl)-1,3-propanedione | | | | | |
| Dioic acid | 0.15 | 0.20 | 0.60 | 0.15 | 1.00 |
| Lipoamide | | 0.4 | | | |
| Uric acid | 0.45 | | 0.40 | 0.25 | 1.00 |
| Tocopherol | 0.1 | | | | 0.20 |
| Biotin | | | 0.05 | | |
| Ethylenediaminetetraacetic acid | 0.1 | | 0.10 | 0.1 | |
| trisodium | | | | | |
| Preservative | q.s. | q.s. | q.s. | q.s. | q.s. |
| Polyacrylic acid | 3.00 | 0.1 | | 0.1 | 0.1 |
| 45% sodium hydroxide solution | q.s. | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |
| Examples | 6 | 7 | 8 | 9 | 10 |
| Glyceryl stearate citrate | | 2.00 | 2.00 | | |
| Glyceryl stearate, self-emulsifying | 5.00 | | | | |
| Polyoxyethylene 20 stearate | | | | | 1.50 |
| Sorbitan monostearate | | | | | 2.50 |
| Stearic acid | | | | 2.50 | |
| Stearyl alcohol | 2.00 | | | | |
| Cetyl alcohol | | | | 3.00 | |
| Cetearyl alcohol | | | | | 2.00 |
| Cetylstearyl alcohol | | 3.00 | 1.00 | | |
| Isohexadecane | | | | | 4.00 |
| C12-15 alkyl benzoate | | 2.00 | 3.00 | | 2.00 |
| Methylglucose sesquistearate | | | | | 0.40 |
| Cetearyl ethylhexanoate | | | | | 4.00 |
| Myristyl myristate | | | | | 1.50 |
| Caprylic/capric triglyceride | 2.00 | | | | |
| Octyldodecanol | 2.00 | 2.00 | | 4.00 | |
| Dicaprylyl ether | | | | | |
| Microcrystalline wax + mineral oil | | | | | 1.00 |
| Liquid paraffin | | 4.00 | 2.00 | | |
| Isopropyl palmitate | | | | | 3.00 |
| Cyclic dimethylpolysiloxane | | | | 0.50 | |
| Dimethicone polydimethylsiloxane | 2.00 | | | | |
| Titanium dioxide | 2.00 | | | | |
| 4-Methylbenzylidenecamphor | 1.00 | | | | |
| 1-(4-tert-Butylphenyl)-3-(4-methoxy- | 0.50 | | | | |
| phenyl)-1,3-propanedione | | | | | |
| Dioic acid | 0.30 | 0.20 | 1.00 | 0.55 | 1.00 |
| Uric acid | 0.70 | 0.40 | 0.30 | 0.20 | |
| Tocopherol | | | | | |
| DL-alpha-tocopheryl acetate | | | | | 0.50 |
| Simmondsia chinensis seed oil | | | | | 1.00 |
| Ethylenediaminetetraacetic acid | | | 0.20 | | |
| trisodium | | | | | |
| Preservative | q.s. | q.s. | q.s. | q.s. | q.s. |
| Xanthan gum | | | 0.20 | | |
| Polyacrylic acid | 0.15 | 0.1 | | 0.05 | 0.30 |
| 45% sodium hydroxide solution | q.s. | q.s. | q.s. | q.s. | q.s. |
| Glycerol | 3.00 | | 3.00 | 5.00 | 2.00 |
| Nitrilotriacetic acid | | | | | 0.10 |
| Lecitin, hydrogenated | | | | | 0.50 |
| Butylene glycol | | 3.00 | | | |
| Ethanol | | 3.00 | | 3.00 | 1.00 |
| Perfume | q.s. | q.s. | q.s. | q.s. | q.s. |
| Water | ad 100 | ad 100 | ad 100 | ad 100 | ad 100 |

| Examples | 11 |
|---|---|
| Stearic acid | 3.50 |
| Cetyl alcohol | 4.50 |
| Cetylstearyl alcohol | 0.50 |
| Octyldodecanol | 6.00 |
| Cyclic dimethylpolysiloxane | 2.00 |
| 4-methylbenzylidene camphor | 1.00 |
| Dioic acid | 0.20 |
| Uric acid | 0.10 |
| Tocopherol | 0.05 |
| Ethylenediaminetetraacetic acid trisodium | 0.20 |
| Preservative | q.s. |
| Polyacrylic acid | 0.05 |
| 45% sodium hydroxide solution | q.s. |
| Glycerol | 3.00 |
| Perfume | q.s. |
| Water | ad 100 |

3. W/O emulsions with antioxidants

| | 1 | 2 | 3 | 4 | 5 |
|---|---|---|---|---|---|
| Cetyldimethicone copolyol | | 2.50 | | 4.00 | |
| Polyglyceryl 2-dipolyhydroxystearate | 5.00 | | | | 4.50 |
| PEG 30 dipolyhydroxystearate | | | 5.00 | | |
| 2-Ethylhexyl methoxycinnamate | | 8.00 | | 5.00 | 4.00 |
| 2,4-bis-(4-(2-Ethylhexyloxy)-2-hydroxyl)- | 2.00 | 2.50 | | 2.00 | 2.50 |
| phenyl)-6-(4-methoxyphenyl)-(1,3,5)- | | | | | |
| triazine | | | | | |
| 1-(4-tert-Butylphenyl)-3-(4-methoxy- | | | 2.00 | 1.00 | |
| phenyl)-1,3-propanedione | | | | | |
| Diethylhexyl butamidotriazone | 3.00 | 1.00 | | | 3.00 |
| Ethylhexyl triazone | | | 3.00 | 4.00 | |
| 4-Methylbenzylidenecamphor | | 2.00 | | 4.00 | 2.00 |
| Octocrylene | 7.00 | 2.50 | 4.00 | | 2.50 |
| Diethylhexyl butamidotriazone | 1.00 | | | 2.00 | |
| Phenylene-1,4,-bis(monosodium, | 1.00 | 2.00 | 0.50 | | |
| 2-benzimidazyl-5,7-disulfonic acid) | | | | | |
| Phenylbenzimidazolsulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Liquid paraffin | | | 10.0 | | 8.00 |
| C12-15 alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.00 | | | 2.00 |
| Shea butter | | | 3.00 | | |
| PVP hexadecene copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| Dioic acid | 0.15 | 0.25 | 0.50 | 0.80 | 1.00 |
| Uric acid | 0.15 | 0.25 | 0.30 | 0.90 | 0.40 |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad 100 | ad 100 | ad 100 | ad 100 | ad 100 |

| | 6 | 7 |
|---|---|---|
| Polyglyceryl 2-dipolyhydroxystearate | 4.00 | 5.00 |
| PEG 30 dipolyhydroxystearate | | |
| Lanolin alcohol | 0.50 | 1.50 |
| Isohexadecane | 1.00 | 2.00 |
| Myristyl myristate | 0.50 | 1.50 |
| Petrolatum | 1.00 | 2.00 |
| 1-(4-tert-Butylphenyl)-3-(4-methoxy-phenyl)-1,3-propanedione | 0.50 | 1.50 |
| 4-Methylbenzylidenecamphor | 1.00 | 3.00 |
| Butylene glycol dicaprylate/dicaprate | 4.00 | 5.00 |
| Shea butter | | 0.50 |
| Butylene glycol | | 6.00 |
| Octoxyglycerol | | 3.00 |
| Glycerol | 5.00 | |
| Tocopherol acetate | 0.50 | 1.00 |
| Dioic acid | 0.50 | 0.40 |
| Uric acid | 0.40 | 0.60 |
| Trisodium EDTA | 0.20 | 0.20 |
| Preservative | q.s. | q.s. |
| Ethanol | | 3.00 |
| Perfume | q.s. | q.s. |
| Water | ad 100 | ad 100 |

4. Hydrodispersions with antioxidants

| | 1 | 2 | 3 | 4 | 5 |
|---|---|---|---|---|---|
| Polyoxyethylene 20 cetylstearyl ether | 1.00 | | | 0.5 | |
| Cetyl alcohol | | | 1.00 | | |
| Sodium polyacrylate | | 0.20 | | 0.30 | |
| Acrylates/C10-30-alkyl acrylate | 0.50 | | 0.40 | 0.10 | 0.10 |
| crosspolymer | | | | | |
| Xanthan gum | | 0.30 | 0.15 | | 0.50 |
| 2-Ethylhexyl methoxycinnamate | | | | 5.00 | 8.00 |
| 2,4-bis(4-(2-Ethylhexyloxy)-2-hydroxyl)- | | 1.50 | | 2.00 | 2.50 |
| phenyl)-6-(4-methoxyphenyl)(1,3,5)- | | | | | |
| triazine | | | | | |
| 1-(4-tert-Butylphenyl)-3-(4-methoxy- | 1.00 | | 2.00 | | |
| phenyl)-1,3-propanedione | | | | | |
| Diethylhexyl butamidotriazone | | 2.00 | | 2.00 | 1.00 |
| Ethylhexyl triazone | 4.00 | | 3.00 | 4.00 | |
| 4-Methylbenzylidenecamphor | 4.00 | 4.00 | | | 2.00 |
| Octocrylene | | 4.00 | 4.00 | | 2.50 |
| Phenylene-1,4-bis(monosodium, | 1.00 | | 0.50 | | 2.00 |
| 2-benzimidazyl-5,7-disulfonic acid) | | | | | |
| Phenylbenzimidazole sulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Dioic acid | 0.3 | 0.15 | 0.40 | 1.00 | 0.30 |
| Uric acid | 0.10 | 0.35 | 0.60 | 0.80 | 0.50 |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

5. Gel cream with antioxidants:

| | |
|---|---|
| Acylate/C10-30 alkyl acrylate | 0.40 |
| crosspolymer | |
| Polyacrylic acid | 0.20 |
| Xanthan gum | 0.10 |
| Cetearyl alcohol | 3.00 |
| C12-15 alkyl benzoate | 4.00 |
| Caprylic/capric triglyceride | 3.00 |
| Cyclic dimethylpolysiloxane | 5.00 |
| Dimethicone polydimethylsiloxane | 1.00 |
| Dioic acid | 0.80 |
| Uric acid | 0.40 |
| Glycerol | 3.00 |
| Sodium hydroxide | q.s. |
| Preservative | q.s. |
| Perfume | q.s. |
| Water | ad 100.0 |
| pH adjusted to 6.0 | |

6. W/O cream with antioxidants:

| | |
|---|---|
| Polyglyceryl 3-diisostearates | 3.50 |
| Glycerol | 3.00 |
| Polyglyceryl 2-dipolyhydroxystearates | 3.50 |
| Dioic acid | 0.60 |
| Uric acid | 0.60 |
| Preservative | q.s. |
| Perfume | q.s. |
| Water | ad 100 |
| Magnesium sulfate | 0.6 |
| Isopropyl stearate | 2.0 |
| Caprylyl ether | 8.0 |
| Cetearyl isononanoate | 6.0 |

7. W/O/W cream with antioxidants:

| | |
|---|---|
| Glyceryl stearate | 3.00 |
| PEG 100 stearate | 0.75 |
| Behenyl alcohol | 2.00 |
| Caprylic/capric triglyceride | 8.0 |
| Octyldodecanol | 5.00 |
| C12-15 alkyl benzoate | 3.00 |
| Dioic acid | 0.70 |
| Uric acid | 0.30 |
| Magnesium sulfate (MgSO₄) | 0.80 |
| Ethylenediaminetetraacetic acid | 0.10 |
| Preservative | q.s. |
| Perfume | q.s. |
| Water | ad 100.0 |
| pH adjusted to 6.0 | |

8. Conditioner shampoo with pearly luster with antioxidants

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Pearlescent agent | 2.0 | 2.0 | 2.0 |
| Uric acid | 0.05 | 0.20 | 0.05 |
| Dioic acid | 0.02 | 0.10 | 0.05 |
| Disodium EDTA | 0.1 | 0.2 | 0.15 |
| Preservative, perfume, thickener, | q.s. | q.s. | q.s. |
| pH adjustment and solubilizer | | | |
| Water, deionized | ad 100 | ad 100 | ad 100 |

The pH is adjusted to 6.

9. Clear conditioner shampoo with antioxidants

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Uric acid | 0.03 | 0.05 | 0.05 |
| Dioic acid | 0.06 | 0.20 | 0.15 |
| Iminodisuccinic acid, Na salt | 0.2 | 0.3 | 0.8 |
| Preservative, perfume, thickener, | q.s. | q.s. | q.s. |
| pH adjustment and solubilizer | | | |
| Water, deionized | ad 100 | ad 100 | ad 100 |

The pH is adjusted to 6.

10. Clear light shampoo with volume effect with antioxidants

| | 1 | 2 | 3 |
|---|---|---|---|
| Sodium laureth sulfate | 10.0 | 10.0 | 10.0 |
| Cocoamidopropylbetaine | 2.5 | 2.5 | 2.5 |
| Uric acid | 0.6 | 0.8 | 0.5 |
| Dioic acid | 0.04 | 0.03 | 0.15 |
| Disodium EDTA | 0.2 | 0.15 | 0.7 |
| Preservative, perfume, thickener, | q.s. | q.s. | q.s. |
| pH adjustment and solubilizer | | | |
| Water, deionized | ad 100 | ad 100 | ad 100 |

The pH is adjusted to 5.5.

11. O/W cream with lipoic acid

| Examples | 1 | 2 | 3 | 4 | 5 |
|---|---|---|---|---|---|
| Glyceryl stearate citrate | | | 2.00 | | 2.00 |
| Glyceryl sterate, self-emulsifying | 4.00 | 3.00 | | | |
| PEG 40 stearate | 1.00 | | | | |
| Polyglyceryl 3-methylglucose distearate | | | | 3.00 | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetylstearyl alcohol | | | | | 2.00 |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Liquid paraffin | 5.00 | 2.00 | | 3.00 | |
| Titanium dioxide | | | 1.00 | | |
| 4-Methylbenzylidenecamphor | | | 1.00 | | |
| 1-(4-tert-Butylphenyl)-3-(4-methoxy- | | | 0.50 | | |
| phenyl)-1,3-propanedione | | | | | |
| Dioic acid | 0.10 | 0.20 | 0.70 | 0.15 | 1.00 |
| Lipoic acid | 0.30 | 0.30 | 0.50 | 0.10 | 1.00 |
| Tocopherol | 0.1 | | | | 0.20 |
| Biotin | | | 0.05 | | |
| Ethylenediaminetetraacetic acid trisodium | 0.1 | | 0.10 | 0.1 | |
| Preservative | q.s. | q.s. | q.s. | q.s. | q.s. |
| Polyacrylic acid | 3.00 | 0.1 | | 0.1 | 0.1 |
| 45% Sodium hydroxide solution | q.s. | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |
| Examples | 6 | 7 | 8 | 9 | 10 |
| Glyceryl stearate citrate | | 2.00 | 2.00 | | |
| Glyceryl stearate, self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetylstearyl alcohol | | 3.00 | 1.00 | | 0.50 |
| C12-15 alkyl benzoate | | 2.00 | 3.00 | | |
| Caprylic/capric triglyceride | 2.00 | | | | |
| Octyldodecanol | 2.00 | 2.00 | | 4.00 | 6.00 |
| Dicaprylyl ether | | | | | |
| Liquid paraffin | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-Methylbenzylidenecamphor | 1.00 | | | | 1.00 |
| 1-(4-tert-Butylphenyl)-3-(4-methoxy- | 0.50 | | | | 0.50 |
| phenyl)-1,3-propanedione | | | | | |
| Dioic acid | 0.30 | 0.10 | 1.00 | 0.50 | 0.10 |
| Lipoic acid | 0.80 | 0.30 | 0.20 | 0.10 | 0.20 |
| Tocopherol | | | | | 0.05 |
| Ethylenediaminetetraacetic acid trisodium | | | 0.20 | | 0.20 |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| 45% Sodium hydroxide solution | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad 100 | ad 100 | ad 100 | ad 100 | ad 100 |

12. W/O emulsions with lipoic acid

| | 1 | 2 | 3 | 4 | 5 |
|---|---|---|---|---|---|
| Cetyl dimethicone copolyol | | 2.50 | | 4.00 | |
| Polyglyceryl 2-dipolyhydroxystearate | 5.00 | | | | 4.50 |
| PEG 30 dipolyhydroxystearate | | | 5.00 | | |
| 2-ethylhexyl methoxycinnamate | | 8.00 | | 5.00 | 4.00 |
| 2,4-bis-(4-(2-Ethylhexyloxy)-2-hydroxyl)- | 2.00 | 2.50 | | 2.00 | 2.50 |
| phenyl)-6-(4-methoxyphenyl)-(1,3,5)- | | | | | |
| triazine | | | | | |
| 1-(4-tert-Butylphenyl)-3-(4-methoxy- | | | 2.00 | 1.00 | |
| phenyl)-1,3-propanedione | | | | | |
| Diethylhexyl butamidotriazone | 3.00 | 1.00 | | | 3.00 |
| Ethylhexyl triazone | | | 3.00 | 4.00 | |
| 4-Methylbenzylidenecamphor | | 2.00 | | 4.00 | 2.00 |
| Octocrylene | 7.00 | 2.50 | 4.00 | | 2.50 |
| Diethylhexyl butamidotriazone | 1.00 | | | 2.00 | |
| Phenylene-1,4-bis(monosodium, | 1.00 | 2.00 | 0.50 | | |
| 2-benzimidazyl-5,7-disulfonic acid | | | | | |
| Phenylbenzimidazole sulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Liquid paraffin | | | 10.0 | | 8.00 |
| C12-15 alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.00 | | | 2.00 |
| Shea butter | | | 3.00 | | |
| PVP hexadecene copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| Dioic acid | 0.10 | 0.20 | 0.50 | 0.90 | 1.00 |
| Lipoic acid | 0.10 | 0.30 | 0.20 | 0.80 | 0.30 |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad 100 | ad 100 | ad 100 | ad 100 | ad 100 |

| | 6 | 7 |
|---|---|---|
| Polyglyceryl 2-dipolyhydroxystearate | 4.00 | 5.00 |
| PEG 30 dipolyhydroxystearate | | |
| Lanolin alcohol | 0.50 | 1.50 |
| Isohexadecane | 1.00 | 2.00 |
| Myristyl myristate | 0.50 | 1.50 |
| Petrolatum | 1.00 | 2.00 |
| 1-(4-tert-Butylphenyl)-3-(4-methoxy-phenyl)-1,3-propanedione | 0.50 | 1.50 |
| 4-Methylbenzylidenecamphor | 1.00 | 3.00 |
| Butylene glycol dicaprylate/dicaprate | 4.00 | 5.00 |
| Shea butter | | 0.50 |
| Butylene glycol | | 6.00 |
| Octoxyglycerol | | 3.00 |
| Glycerol | 5.00 | |
| Tocopherol acetate | 0.50 | 1.00 |
| Dioic acid | 0.10 | 0.70 |
| Lipoic acid | 1.00 | 0.60 |
| Trisodium EDTA | 0.20 | 0.20 |
| Preservative | q.s. | q.s. |
| Ethanol | | 3.00 |
| Perfume | q.s. | q.s. |
| Water | ad 100 | ad 100 |

13. Hydrodispersions with lipoic acid

| | 1 | 2 | 3 | 4 | 5 |
|---|---|---|---|---|---|
| Polyoxyethylene 20 cetylstearyl ether | 1.00 | | | 0.5 | |
| Cetyl alcohol | | | 1.00 | | |
| Sodium polyacrylate | | 0.20 | | 0.30 | |
| Acrylates/C10-30-alkyl acrylate | 0.50 | | 0.40 | 0.10 | 0.10 |
| crosspolymer | | | | | |
| Xanthan gum | | 0.30 | 0.15 | | 0.50 |
| 2-Ethylhexyl methoxycinnamate | | | | 5.00 | 8.00 |
| 2,4-bis(4-(2-Ethylhexyloxy)-2-hydroxyl)- | | 1.50 | | 2.00 | 2.50 |
| phenyl)-6-(4-methoxyphenyl)-(1,3,5)- | | | | | |
| triazine | | | | | |
| 1-(4-tert-Butylphenyl)-3-(4-methoxy- | 1.00 | | 2.00 | | |
| phenyl)-1,3-propanedione | | | | | |
| Diethylhexyl butamidotriazone | | 2.00 | | 2.00 | 1.00 |
| Ethylhexyl triazone | 4.00 | | 3.00 | 4.00 | |
| 4-Methylbenzylidenecamphor | 4.00 | 4.00 | | | 2.00 |
| Octocrylene | | 4.00 | 4.00 | | 2.50 |
| Phenylene-1,4-bis(monosodium, | 1.00 | | 0.50 | | 2.00 |
| 2-benzimidazyl-5,7-disulfonic acid) | | | | | |
| Phenylbenzimidazole sulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Dioic acid | 0.3 | 0.10 | 0.50 | 1.00 | 0.20 |
| Lipoic acid | 0.10 | 0.30 | 1.00 | 0.70 | 0.50 |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

14. Gel cream with lipoic acid:

| | |
|---|---|
| Acrylate/C10-30 alkyl acrylate crosspolymer | 0.40 |
| Polyacrylic acid | 0.20 |
| Xanthan gum | 0.10 |
| Cetearyl alcohol | 3.00 |
| C12-15 alkyl benzoate | 4.00 |
| Caprylic/capric triglyceride | 3.00 |
| Cyclic dimethylpolysiloxane | 5.00 |
| Dimethicone polydimethylsiloxane | 1.00 |
| Dioic acid | 1.00 |
| Lipoic acid | 0.50 |
| Glycerol | 3.00 |
| Sodium hydroxide | q.s. |
| Preservative | q.s. |
| Perfume | q.s. |
| Water | ad 100.0 |
| pH adjusted to 6.0 | |

15. W/O cream with lipoic acid

| | |
|---|---|
| Polyglyceryl 3-diisostearates | 3.50 |
| Glycerol | 3.00 |
| Polyglyceryl 2-dipolyhydroxystearates | 3.50 |
| Dioic acid | 0.70 |
| Lipoic acid | 0.70 |
| Preservative | q.s. |
| Perfume | q.s. |
| Water | ad 100.0 |
| Magnesium sulfate | 0.6 |
| Isopropyl stearate | 2.0 |
| Caprylyl ether | 8.0 |
| Cetearyl isononanoate | 6.0 |

16. Conditioner shampoo with pearly luster and with lipoic acid

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Pearlescent agent | 2.0 | 2.0 | 2.0 |
| Lipoic acid | 0.04 | 0.08 | 0.02 |
| Dioic acid | 0.02 | 0.05 | 0.015 |
| Disodium EDTA | 0.1 | 0.2 | 0.15 |
| Preservative, perfume, thickener, | q.s. | q.s. | q.s. |
| pH adjustment and solubilizer | | | |
| Water, deionized | ad 100.0 | ad 100.0 | ad 100.0 |

The pH is adjusted to 6.

17. Clear conditioner shampoo with lipoic acid

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Lipoic acid | 0.01 | 0.1 | 0.05 |
| Dioic acid | 0.04 | 0.10 | 0.07 |
| Iminodisuccinic acid, Na salt | 0.2 | 0.3 | 0.8 |
| Preservative, perfume, thickener, | q.s. | q.s. | q.s. |
| pH adjustment and solubilizer | | | |
| Water, deionized | ad 100.0 | ad 100.0 | ad 100.0 |

The pH is adjusted to 6.

18. Clear light shampoo with volume effect and with lipoic acid

| | 1 | 2 | 3 |
|---|---|---|---|
| Sodium laureth sulfate | 10.0 | 10.0 | 10.0 |
| Cocoamidopropylbetaine | 2.5 | 2.5 | 2.5 |
| Lipoic acid | 0.3 | 0.4 | 0.6 |
| Dioic acid | 0.05 | 0.1 | 0.01 |
| Disodium EDTA | 0.2 | 0.15 | 0.7 |
| Preservative, perfume, thickener, | q.s. | q.s. | q.s. |
| pH adjustment and solubilizer | | | |
| Water, deionized | ad 100.0 | ad 100.0 | ad 100.0 |

The pH is adjusted to 5.5.

19. O/W cream with carnosine

| Examples | 1 | 2 | 3 | 4 | 5 |
|---|---|---|---|---|---|
| Glyceryl stearate citrate | | | 2.00 | | 2.00 |
| Glyceryl sterate, self-emulsifying | 4.00 | 3.00 | | | |
| PEG 40 stearate | 1.00 | | | | |
| Polyglyceryl 3-methylglucose distearate | | | | 3.00 | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetylstearyl alcohol | | | | | 2.00 |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Liquid paraffin | 5.00 | 2.00 | | 3.00 | |
| Titanium dioxide | | | 1.00 | | |
| 4-Methylbenzylidenecamphor | | | 1.00 | | |
| 1-(4-tert-Butylphenyl)-3-(4-methoxy- | | | 0.50 | | |
| phenyl)-1,3-propanedione | | | | | |
| Dioic acid | 0.10 | 0.20 | 0.70 | 0.15 | 1.00 |
| Carnosine | 0.30 | 0.30 | 0.50 | 0.10 | 1.00 |
| Tocopherol | 0.1 | | | | 0.20 |
| Biotin | | | 0.05 | | |
| Ethylenediaminetetraacetic acid trisodium | 0.1 | | 0.10 | 0.1 | |
| Preservative | q.s. | q.s. | q.s. | q.s. | q.s. |
| Polyacrylic acid | 3.00 | 0.1 | | 0.1 | 0.1 |
| 45% sodium hydroxide solution | q.s. | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |
| Examples | 6 | 7 | 8 | 9 | 10 |
| Glyceryl stearate citrate | | 2.00 | 2.00 | | |
| Glyceryl sterate, self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetylstearyl alcohol | | 3.00 | 1.00 | | 0.50 |
| C12-15 alkyl benzoate | | 2.00 | 3.00 | | |
| Caprylic/capric triglyceride | 2.00 | | | | |
| Octyldodecanol | 2.00 | 2.00 | | 4.00 | 6.00 |
| Dicapryl ether | | | | | |
| Liquid paraffin | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-Methylbenzylidenecamphor | 1.00 | | | | 1.00 |
| 1-(4-tert-Butylphenyl)-3-(4-methoxy- | 0.50 | | | | 0.50 |
| phenyl)-1,3-propanedione | | | | | |
| Dioic acid | 0.30 | 0.10 | 1.00 | 0.50 | 0.10 |
| Carnosine | 0.80 | 0.30 | 0.20 | 0.10 | 0.20 |
| Tocopherol | | | | | 0.05 |
| Ethylenediaminetetraacetic acid trisodium | | | 0.20 | | 0.20 |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| 45% Sodium hydroxide solution | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad 100 | ad 100 | ad 100 | ad 100 | ad 100 |

20. W/O emulsions with carnosine

| | 1 | 2 | 3 | 4 | 5 |
|---|---|---|---|---|---|
| Cetyldimethicone copolyol | | 2.50 | | 4.00 | |
| Polyglyceryl 2-dipolyhydroxystearate | 5.00 | | | | 4.50 |
| PEG 30 dipolyhydroxystearate | | | 5.00 | | |
| 2-Ethylhexyl methoxycinnamate | | 8.00 | | 5.00 | 4.00 |
| 2,4-bis-(4-(2-Ethylhexyloxy)-2-hydroxyl)- | 2.00 | 2.50 | | 2.00 | 2.50 |
| phenyl)-6-(4-methoxyphenyl)-(1,3,5)- | | | | | |
| triazine | | | | | |
| 1-(4-tert-Butylphenyl)-3-(4-methoxy- | | | 2.00 | 1.00 | |
| phenyl)-1,3-propanedione | | | | | |
| Diethylhexyl butamidotriazone | 3.00 | 1.00 | | | 3.00 |
| Ethylhexyl triazone | | | 3.00 | 4.00 | |
| 4-Methylbenzylidenecamphor | | 2.00 | | 4.00 | 2.00 |
| Octocrylene | 7.00 | 2.50 | 4.00 | | 2.50 |
| Diethylhexyl butamidotriazone | 1.00 | | | 2.00 | |
| Phenylene-1,4,-bis(monosodium, | 1.00 | 2.00 | 0.50 | | |
| 2-benzimidazyl-5,7-disulfonic acid) | | | | | |
| Phenylbenzimidazolsulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Liquid paraffin | | | 10.0 | | 8.00 |
| C12-15 alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.00 | | | 2.00 |
| Shea butter | | | 3.00 | | |
| PVP hexadecene copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| Dioic acid | 0.10 | 0.20 | 0.50 | 0.90 | 1.00 |
| Carnosine | 0.10 | 0.30 | 0.20 | 0.80 | 0.30 |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad 100 | ad 100 | ad 100 | ad 100 | ad 100 |

| | 6 | 7 |
|---|---|---|
| Polyglyceryl 2-dipolyhydroxystearate | 4.00 | 5.00 |
| PEG 30 dipolyhydroxystearate | | |
| Lanolin alcohol | 0.50 | 1.50 |
| Isohexadecane | 1.00 | 2.00 |
| Myristyl myristate | 0.50 | 1.50 |
| Petrolatum | 1.00 | 2.00 |
| 1-(4-tert-Butylphenyl)-3-(4-methoxy-phenyl)-1,3-propanedione | 0.50 | 1.50 |
| 4-Methylbenzylidenecamphor | 1.00 | 3.00 |
| Butylene glycol dicaprylate/dicaprate | 4.00 | 5.00 |
| Shea butter | | 0.50 |
| Butylene glycol | | 6.00 |
| Octoxyglycerol | | 3.00 |
| Glycerol | 5.00 | |
| Tocopherol acetate | 0.50 | 1.00 |
| Dioic acid | 0.10 | 0.70 |
| Carnosine | 1.00 | 0.60 |
| Trisodium EDTA | 0.20 | 0.20 |
| Preservative | q.s. | q.s. |
| Ethanol | | 3.00 |
| Perfume | q.s. | q.s. |
| Water | ad 100 | ad 100 |

21. Hydrodispersions with carnosine

| | 1 | 2 | 3 | 4 | 5 |
|---|---|---|---|---|---|
| Polyoxyethylene 20 cetylstearyl ether | 1.00 | | | 0.5 | |
| Cetyl alcohol | | | 1.00 | | |
| Sodium polyacrylate | | 0.20 | | 0.30 | |
| Acrylates/C10-30-alkyl acrylate | 0.50 | | 0.40 | 0.10 | 0.10 |
| crosspolymer | | | | | |
| Xanthan gum | | 0.30 | 0.15 | | 0.50 |
| 2-Ethylhexyl methoxycinnamate | | | | 5.00 | 8.00 |
| 2,4-bis(4-(2-Ethylhexyloxy)-2-hydroxyl)- | | 1.50 | | 2.00 | 2.50 |
| phenyl)-6-(4-methoxyphenyl)(1,3,5)- | | | | | |
| triazine | | | | | |
| 1-(4-tert-Butylphenyl)-3-(4-methoxy- | 1.00 | | 2.00 | | |
| phenyl)-1,3-propanedione | | | | | |
| Diethylhexyl butamidotriazone | | 2.00 | | 2.00 | 1.00 |
| Ethylhexyl triazone | 4.00 | | 3.00 | 4.00 | |
| 4-Methylbenzylidenecamphor | 4.00 | 4.00 | | | 2.00 |
| Octocrylene | | 4.00 | 4.00 | | 2.50 |
| Phenylene-1,4-bis(monosodium, | 1.00 | | 0.50 | | 2.00 |
| 2-benzimidazyl-5,7-disulfonic acid) | | | | | |
| Phenylbenzimidazole sulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Dioic acid | 0.3 | 0.10 | 0.50 | 1.00 | 0.20 |
| Carnosine | 0.10 | 0.30 | 1.00 | 0.70 | 0.50 |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

22. Gel cream with carnosine:

| | |
|---|---|
| Acylate/C10-30 alkyl acrylate crosspolymer | 0.40 |
| Polyacrylic acid | 0.20 |
| Xanthan gum | 0.10 |
| Cetearyl alcohol | 3.00 |
| C12-15 alkyl benzoate | 4.00 |
| Caprylic/capric triglyceride | 3.00 |
| Cyclic dimethylpolysiloxane | 5.00 |
| Dimethicone polydimethylsiloxane | 1.00 |
| Dioic acid | 1.00 |
| Carnosine | 0.50 |
| Glycerol | 3.00 |
| Sodium hydroxide | q.s. |
| Preservative | q.s. |
| Perfume | q.s. |
| Water | ad 100.0 |
| pH adjusted to 6.0 | |

23. W/O cream with carnosine

| | |
|---|---|
| Polyglyceryl 3-diisostearates | 3.50 |
| Glycerol | 3.00 |
| Polyglyceryl 2-dipolyhydroxystearates | 3.50 |
| Dioic acid | 0.70 |
| Carnosine | 0.70 |
| Preservative | q.s. |
| Perfume | q.s. |
| Water | ad 100.0 |
| Magnesium sulfate | 0.6 |
| Isopropyl stearate | 2.0 |
| Caprylyl ether | 8.0 |
| Cetearyl isononanoate | 6.0 |

24. Conditioner shampoo with pearly luster and with carnosine

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Pearlescent agent | 2.0 | 2.0 | 2.0 |
| Carnosine | 0.15 | 0.2 | 0.05 |
| Dioic acid | 0.03 | 0.05 | 0.02 |
| Disodium EDTA | 0.1 | 0.2 | 0.15 |
| Preservative, perfume, thickener, | q.s. | q.s. | q.s. |
| pH adjustment and solubilizer | | | |
| Water, deionized | ad 100.0 | ad 100.0 | ad 100.0 |

The pH is adjusted to 6.

25. Clear conditioner shampoo with carnosine

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Carnosine | 0.02 | 0.15 | 0.05 |
| Dioic acid | 0.05 | 0.15 | 0.05 |
| Iminodisuccinic acid, Na salt | 0.2 | 0.3 | 0.8 |
| Preservative, perfume, thickener, | q.s. | q.s. | q.s. |
| pH adjustment and solubilizer | | | |
| Water, deionized | ad 100.0 | ad 100.0 | ad 100.0 |

The pH is adjusted to 6.

26. Clear light shampoo with volume effect and with carnosine

| | 1 | 2 | 3 |
|---|---|---|---|
| Sodium laureth sulfate | 10.0 | 10.0 | 10.0 |
| Cocoamidopropylbetaine | 2.5 | 2.5 | 2.5 |
| Carnosine | 0.3 | 0.3 | 0.7 |
| Dioic acid | 0.07 | 0.15 | 0.02 |
| Disodium EDTA | 0.2 | 0.15 | 0.7 |
| Preservative, perfume, thickener, | q.s. | q.s. | q.s. |
| pH adjustment and solubilizer | | | |
| Water, deionized | ad 100.0 | ad 100.0 | ad 100.0 |

The pH is adjusted to 5.5.

27. O/W cream with bioquinones

| Examples | 1 | 2 | 3 | 4 | 5 |
|---|---|---|---|---|---|
| Glyceryl stearate citrate | | | 2.00 | | 2.00 |
| Glyceryl sterate, self-emulsifying | 4.00 | 3.00 | | | |
| PEG 40 stearate | 1.00 | | | | |
| Polyglyceryl 3-methylglucose distearate | | | | 3.00 | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetylstearyl alcohol | | | | | 2.00 |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Liquid paraffin | 5.00 | 2.00 | | 3.00 | |
| Titanium dioxide | | | 1.00 | | |
| 4-Methylbenzylidenecamphor | | | 1.00 | | |
| 1-(4-tert-Butylphenyl)-3-(4-methoxy- | | | 0.50 | | |
| phenyl)-1,3-propanedione | | | | | |
| Dioic acid | 0.10 | 0.20 | 0.70 | 0.15 | 1.00 |
| Ubiquinone (Q10) | 0.30 | 0.30 | 0.50 | 0.10 | 1.00 |
| Tocopherol | 0.1 | | | | 0.20 |
| Biotin | | | 0.05 | | |
| Ethylenediaminetetraacetic acid trisodium | 0.1 | | 0.10 | 0.1 | |
| Preservative | q.s. | q.s. | q.s. | q.s. | q.s. |
| Polyacrylic acid | 3.00 | 0.1 | | 0.1 | 0.1 |
| 45% sodium hydroxide solution | q.s. | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |
| Examples | 6 | 7 | 8 | 9 | 10 |
| Glyceryl stearate citrate | | 2.00 | 2.00 | | |
| Glyceryl sterate, self-emulsifying | 5.00 | | | | |
| Stearic acid | | | | 2.50 | 3.50 |
| Stearyl alcohol | 2.00 | | | | |
| Cetyl alcohol | | | | 3.00 | |
| Cetearyl alcohol | | | | 3.00 | 4.50 |
| Cetylstearyl alcohol | | 3.00 | 1.00 | | 0.50 |
| C12-15 alkyl benzoate | | 2.00 | 3.00 | | |
| Caprylic/capric triglyceride | 2.00 | | | | |
| Octyldodecanol | 2.00 | 2.00 | | 4.00 | 6.00 |
| Dicaprylyl ether | | | | | |
| Liquid paraffin | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-Methylbenzylidenecamphor | 1.00 | | | | 1.00 |
| 1-(4-tert-Butylphenyl)-3-(4-methoxy- | 0.50 | | | | 0.50 |
| phenyl)-1,3-propanedione | | | | | |
| Dioic acid | 0.30 | 0.10 | 1.00 | 0.50 | 0.10 |
| Ubiquinone (Q10) | 0.80 | 0.30 | 0.20 | 0.10 | 0.20 |
| Tocopherol | | | | | 0.05 |
| Ethylenediaminetetraacetic acid trisodium | | | 0.20 | | 0.20 |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| 45% sodium hydroxide solution | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad 100 | ad 100 | ad 100 | ad 100 | ad 100 |

28. W/O emulsions with bioquinones

| | 1 | 2 | 3 | 4 | 5 |
|---|---|---|---|---|---|
| Cetyldimethicone copolyol | | 2.50 | | 4.00 | |
| Polyglyceryl 2-dipolyhydroxystearate | 5.00 | | | | 4.50 |
| PEG 30 dipolyhydroxystearate | | | 5.00 | | |
| 2-Ethylhexyl methoxycinnamate | | 8.00 | | 5.00 | 4.00 |
| 2,4-bis-(4-(2-Ethylhexyloxy)-2-hydroxyl)- | 2.00 | 2.50 | | 2.00 | 2.50 |
| phenyl)-6-(4-methoxyphenyl)-(1,3,5)- | | | | | |
| triazine | | | | | |
| 1-(4-tert-Butylphenyl)-3-(4-methoxy- | | | 2.00 | 1.00 | |
| phenyl)-1,3-propanedione | | | | | |
| Diethylhexyl butamidotriazone | 3.00 | 1.00 | | | 3.00 |
| Ethylhexyl triazone | | | 3.00 | 4.00 | |
| 4-Methylbenzylidenecamphor | | 2.00 | | 4.00 | 2.00 |
| Octocrylene | 7.00 | 2.50 | 4.00 | | 2.50 |
| Diethylhexyl butamidotriazone | 1.00 | | | 2.00 | |
| Phenylene-1,4,-bis(monosodium, | 1.00 | 2.00 | 0.50 | | |
| 2-benzimidazyl-5,7-disulfonic acid) | | | | | |
| Phenylbenzimidazolsulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Liquid paraffin | | | 10.0 | | 8.00 |
| C12-15 alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.00 | | | 2.00 |
| Shea butter | | | 3.00 | | |
| PVP hexadecene copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| Dioic acid | 0.10 | 0.20 | 0.50 | 0.90 | 1.00 |
| Ubiquinone (Q10) | 0.10 | 0.30 | 0.20 | 0.80 | 0.30 |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad 100 | ad 100 | ad 100 | ad 100 | ad 100 |

| | 6 | 7 |
|---|---|---|
| Polyglyceryl 2-dipolyhydroxystearate | 4.00 | 5.00 |
| PEG 30 dipolyhydroxystearate | | |
| Lanolin alcohol | 0.50 | 1.50 |
| Isohexadecane | 1.00 | 2.00 |
| Myristyl myristate | 0.50 | 1.50 |
| Petrolatum | 1.00 | 2.00 |
| 1-(4-tert-Butylphenyl)-3-(4-methoxy-phenyl)-1,3-propanedione | 0.50 | 1.50 |
| 4-Methylbenzylidenecamphor | 1.00 | 3.00 |
| Butylene glycol dicaprylate/dicaprate | 4.00 | 5.00 |
| Shea butter | | 0.50 |
| Butylene glycol | | 6.00 |
| Octoxyglycerol | | 3.00 |
| Glycerol | 5.00 | |
| Tocopherol acetate | 0.50 | 1.00 |
| Dioic acid | 0.10 | 0.70 |
| Ubiquinone (Q10) | 1.00 | 0.60 |
| Trisodium EDTA | 0.20 | 0.20 |
| Preservative | q.s. | q.s. |
| Ethanol | | 3.00 |
| Perfume | q.s. | q.s. |
| Water | ad 100 | ad 100 |

29. Hydrodispersions with bioquinones

| | 1 | 2 | 3 | 4 | 5 |
|---|---|---|---|---|---|
| Polyoxyethylene 20 cetylstearyl ether | 1.00 | | | 0.5 | |
| Cetyl alcohol | | | 1.00 | | |
| Sodium polyacrylate | | 0.20 | | 0.30 | |
| Acrylates/C10-30-alkyl acrylate crosspolymer | 0.50 | | 0.40 | 0.10 | 0.10 |
| Xanthan gum | | 0.30 | 0.15 | | 0.50 |
| 2-Ethylhexyl methoxycinnamate | | | | 5.00 | 8.00 |
| 2,4-bis(4-(2-Ethylhexyloxy)-2-hydroxyl)- | | 1.50 | | 2.00 | 2.50 |
| phenyl)-6-(4-methoxyphenyl)(1,3,5)-triazine | | | | | |
| 1-(4-tert-Butylphenyl)-3-(4-methoxy- | 1.00 | | 2.00 | | |
| phenyl)-1,3-propanedione | | | | | |
| Diethylhexyl butamidotriazone | | 2.00 | | 2.00 | 1.00 |
| Ethylhexyl triazone | 4.00 | | 3.00 | 4.00 | |
| 4-Methylbenzylidenecamphor | 4.00 | 4.00 | | | 2.00 |
| Octocrylene | | 4.00 | 4.00 | | 2.50 |
| Phenylene-1,4-bis(monosodium, | 1.00 | | 0.50 | | 2.00 |
| 2-benzimidazyl-5,7-disulfonic acid) | | | | | |
| Phenylbenzimidazole sulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Dioic acid | 0.3 | 0.10 | 0.50 | 1.00 | 0.20 |
| Ubiquinone (Q10) | 0.10 | 0.30 | 1.00 | 0.70 | 0.50 |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

30. Gel cream with bioquinones:

| | |
|---|---|
| Acylate/C10-30 alkyl acrylate crosspolymer | 0.40 |
| Polyacrylic acid | 0.20 |
| Xanthan gum | 0.10 |
| Cetearyl alcohol | 3.00 |
| C12-15 alkyl benzoate | 4.00 |
| Caprylic/capric triglyceride | 3.00 |
| Cyclic dimethylpolysiloxane | 5.00 |
| Dimethicone polydimethylsiloxane | 1.00 |
| Dioic acid | 1.00 |
| Ubiquinone (Q10) | 0.50 |
| Glycerol | 3.00 |
| Sodium hydroxide | q.s. |
| Preservative | q.s. |
| Perfume | q.s. |
| Water | ad 100.0 |
| pH adjusted to 6.0 | |

31. W/O cream with bioquinones

| | |
|---|---|
| Polyglyceryl 3-diisostearates | 3.50 |
| Glycerol | 3.00 |
| Polyglyceryl 2-dipolyhydroxystearates | 3.50 |
| Dioic acid | 0.70 |
| Ubiquinone (Q10) | 0.70 |
| Preservative | q.s. |
| Perfume | q.s. |
| Water | ad 100.0 |
| Magnesium sulfate | 0.6 |
| Isopropyl stearate | 2.0 |
| Caprylyl ether | 8.0 |
| Cetearyl isononanoate | 6.0 |

32. Conditioner shampoo with pearly luster and with bioquinones

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Pearlescent agent | 2.0 | 2.0 | 2.0 |
| Ubiquinone (Q10) | 0.06 | 0.15 | 0.01 |
| Dioic acid | 0.03 | 0.05 | 0.02 |
| Disodium EDTA | 0.1 | 0.2 | 0.15 |
| Preservative, perfume, thickener, | q.s. | q.s. | q.s. |
| pH adjustment and solubilizer | | | |
| Water, deionized | ad 100.0 | ad 100.0 | ad 100.0 |

The ph is adjusted to 6.

33. Clear conditioner shampoo with bioquinones

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Ubiquinone (Q10) | 0.02 | 0.05 | 0.05 |
| Dioic acid | 0.05 | 0.15 | 0.1 |
| Iminodisuccinic acid, Na salt | 0.2 | 0.3 | 0.8 |
| Preservative, perfume, thickener, | q.s. | q.s. | q.s. |
| pH adjustment and solubilizer | | | |
| Water, deionized | ad 100.0 | ad 100.0 | ad 100.0 |

The pH is adjusted to 6.

34. Clear light shampoo with volume effect and with bioquinones

| | 1 | 2 | 3 |
|---|---|---|---|
| Sodium laureth sulfate | 10.0 | 10.0 | 10.0 |
| Cocoamidopropylbetaine | 2.5 | 2.5 | 2.5 |
| Ubiquinone (Q10) | 0.5 | 0.7 | 0.3 |
| Dioic acid | 0.05 | 0.01 | 0.15 |
| Disodium EDTA | 0.2 | 0.15 | 0.7 |
| Preservative, perfume, thickener, | q.s. | q.s. | q.s. |
| pH adjustment and solubilizer | | | |
| Water, deionized | ad 100.0 | ad 100.0 | ad 100.0 |

The pH is adjusted to 5.5.

35. PIT emulsions with tyrosine sulfate

| | 1 | 2 | 3 | 4 | 5 |
|---|---|---|---|---|---|
| Glycerol monostearate, self-emulsifying | 0.50 | | 3.00 | 2.00 | 4.00 |
| Polyoxyethylene 12 cetylstearyl ether | | 5.00 | | 1.00 | 1.50 |
| Polyoxyethylene 20 cetylstearyl ether | | | | 2.00 | |
| Polyoxyethylene 30 cetylstearyl ether | 5.00 | | 1.00 | | |
| Stearyl alcohol | | | 3.00 | | 0.50 |
| Cetyl alcohol | 2.50 | 1.00 | | 1.50 | |
| 2-Ethylhexyl methoxycinnamate | | | | 5.00 | 8.00 |
| 2,4-bis(4-(2-Ethylhexyloxy)-2-hydroxyl)- | | 1.50 | | 2.00 | 2.50 |
| phenyl)-6-(4-methoxyphenyl)-(1,3,5)-triazine | | | | | |
| 1-(4-tert-butylphenyl)-3-(4- | | | 2.00 | | |
| methoxyphenyl)-1,3-propanedione | | | | | |
| Diethylhexyl butamidotriazone | 1.00 | 2.00 | | 2.00 | |
| Ethylhexyltriazone | 4.00 | | 3.00 | 4.00 | |
| 4-Methylbenzylidenecamphor | | 4.00 | | | 2.00 |
| Octocrylene | | 4.00 | | | 2.50 |
| Phenylene-1,4-bis(monosodium, | | | 0.50 | | 1.50 |
| 2-benzimidazyl-5,7-disulfonic acid | | | | | |
| Phenylbenzimidazole sulfonic acid | 0.50 | | | 3.00 | |
| C12-15 alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethylpolysiloxane | 2.00 | | | 0.50 | 0.50 |
| Shea butter | | 2.00 | | | 0.50 |
| PVP hexadecene copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| Dioic acid | 0.10 | 1.00 | 0.20 | 0.50 | 0.10 |
| Tyrosine sulfate | 0.80 | 0.10 | 0.60 | 0.20 | 0.30 |
| Alpha-glucosylrutin | 0.10 | | 0.20 | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s: |
| Water | ad 100 | ad 100 | ad 100 | ad 100 | ad 100 |

36. O/W creams with tyrosine sulfate

| Examples | 1 | 2 | 3 | 4 | 5 |
|---|---|---|---|---|---|
| Glyceryl stearate citrate | | | 2.00 | | 2.00 |
| Glyceryl sterate, self-emulsifying | 4.00 | 3.00 | | | |
| PEG 40 stearate | 1.00 | | | | |
| Polyglyceryl 3-methylglucose distearate | | | | 3.00 | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetylstearyl alcohol | | | | | 2.00 |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Liquid paraffin | 5.00 | 2.00 | | 3.00 | |
| Titanium dioxide | | | 1.00 | | |
| 4-Methylbenzylidenecamphor | | | 1.00 | | |
| 1-(4-tert-Butylphenyl)-3-(4-methoxy- | | | 0.50 | | |
| phenyl)-1,3-propanedione | | | | | |
| Dioic acid | 0.10 | 0.20 | 0.70 | 0.15 | 1.00 |
| Tyrosine sulfate | 0.30 | 0.30 | 0.50 | 0.10 | 1.00 |
| Tocopherol | 0.1 | | | | 0.20 |
| Biotin | | | 0.05 | | |
| Ethylenediaminetetraacetic acid trisodium | 0.1 | | 0.10 | 0.1 | |
| Preservative | q.s. | q.s. | q.s. | q.s. | q.s. |
| Polyacrylic acid | 3.00 | 0.1 | | 0.1 | 0.1 |
| 45% sodium hydroxide solution | q.s. | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |
| Examples | 6 | 7 | 8 | 9 | 10 |
| Glyceryl stearate citrate | | 2.00 | 2.00 | | |
| Glyceryl sterate, self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetylstearyl alcohol | | 3.00 | 1.00 | | 0.50 |
| C12-15 alkyl benzoate | | 2.00 | 3.00 | | |
| Caprylic/capric triglyceride | 2.00 | | | | |
| Octyldodecanol | 2.00 | 2.00 | | 4.00 | 6.00 |
| Dicaprylyl ether | | | | | |
| Liquid paraffin | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-Methylbenzylidenecamphor | 1.00 | | | | 1.00 |
| 1-(4-tert-Butylphenyl)-3-(4-methoxy- | 0.50 | | | | 0.50 |
| phenyl)-1,3-propanedione | | | | | |
| Dioic acid | 0.30 | 0.10 | 1.00 | 0.50 | 0.10 |
| Tyrosine sulfate | 0.80 | 0.30 | 0.20 | 0.10 | 0.20 |
| Tocopherol | | | | | 0.05 |
| Ethylenediaminetetraacetic acid trisodium | | | 0.20 | | 0.20 |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| 45% sodium hydroxide solution | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad 100 | ad 100 | ad 100 | ad 100 | ad 100 |

37. W/O emulsions with tyrosine sulfate

| | 1 | 2 | 3 | 4 | 5 |
|---|---|---|---|---|---|
| Cetyldimethicone copolyol | | 2.50 | | 4.00 | |
| Polyglyceryl 2-dipolyhydroxystearate | 5.00 | | | | 4.50 |
| PEG 30 dipolyhydroxystearate | | | 5.00 | | |
| 2-Ethylhexyl methoxycinnamate | | 8.00 | | 5.00 | 4.00 |
| 2,4-bis-(4-(2-Ethylhexyloxy)-2-hydroxyl)- | 2.00 | 2.50 | | 2.00 | 2.50 |
| phenyl-6)-(4-methoxyphenyl)-(1,3,5)- | | | | | |
| triazine | | | | | |
| 1-(4-tert-Butylphenyl)-3-(4-methoxy- | | | 2.00 | 1.00 | |
| phenyl)-1,3-propanedione | | | | | |
| Diethylhexyl butamidotriazone | 3.00 | 1.00 | | | 3.00 |
| Ethylhexyl triazone | | | 3.00 | 4.00 | |
| 4-Methylbenzylidenecamphor | | 2.00 | | 4.00 | 2.00 |
| Octocrylene | 7.00 | 2.50 | 4.00 | | 2.50 |
| Diethylhexyl butamidotriazone | 1.00 | | | 2.00 | |
| Phenylene-1,4,-bis(monosodium, | 1.00 | 2.00 | 0.50 | | |
| 2-benzimidazyl-5,7-disulfonic acid) | | | | | |
| Phenylbenzimidazolsulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Liquid paraffin | | | 10.0 | | 8.00 |
| C12-15 alkyl benzoate | | | | 9.00 | |
| Dicapryl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.00 | | | 2.00 |
| Shea butter | | | 3.00 | | |
| PVP hexadecene copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| Dioic acid | 0.10 | 0.20 | 0.50 | 0.90 | 1.00 |
| Tyrosine sulfate | 0.10 | 0.30 | 0.20 | 0.80 | 0.30 |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad 100 | ad 100 | ad 100 | ad 100 | ad 100 |

| | 6 | 7 |
|---|---|---|
| Polyglyceryl 2-dipolyhydroxystearate | 4.00 | 5.00 |
| PEG 30 dipolyhydroxystearate | | |
| Lanolin alcohol | 0.50 | 1.50 |
| Isohexadecane | 1.00 | 2.00 |
| Myristyl myristate | 0.50 | 1.50 |
| Petrolatum | 1.00 | 2.00 |
| 1-(4-tert-Butylphenyl)-3-(4-methoxy-phenyl)-1,3-propanedione | 0.50 | 1.50 |
| 4-Methylbenzylidenecamphor | 1.00 | 3.00 |
| Butylene glycol dicaprylate/dicaprate | 4.00 | 5.00 |
| Shea butter | | 0.50 |
| Butylene glycol | | 6.00 |
| Octoxyglycerol | | 3.00 |
| Glycerol | 5.00 | |
| Tocopherol acetate | 0.50 | 1.00 |
| Dioic acid | 0.10 | 0.70 |
| Tyrosine sulfate | 1.00 | 0.60 |
| Trisodium EDTA | 0.20 | 0.20 |
| Preservative | q.s. | q.s. |
| Ethanol | | 3.00 |
| Perfume | q.s. | q.s. |
| Water | ad 100 | ad 100 |

38. Hydrodispersions with tyrosine sulfate

| | 1 | 2 | 3 | 4 | 5 |
|---|---|---|---|---|---|
| Polyoxyethylene 20 cetylstearyl ether | 1.00 | | | 0.5 | |
| Cetyl alcohol | | | 1.00 | | |
| Sodium polyacrylate | | 0.20 | | 0.30 | |
| Acrylates/C10-30-alkyl acrylate crosspolymer | 0.50 | | 0.40 | 0.10 | 0.10 |
| Xanthan gum | | 0.30 | 0.15 | | 0.50 |
| 2-Ethylhexyl methoxycinnamate | | | | 5.00 | 8.00 |
| 2,4-bis(4-(2-Ethylhexyloxy)-2-hydroxyl)- | | 1.50 | | 2.00 | 2.50 |
| phenyl)-6-(4-methoxyphenyl)(1,3,5)-triazine | | | | | |
| 1-(4-tert-Butylphenyl)-3-(4-methoxy- | 1.00 | | 2.00 | | |
| phenyl)-1,3-propanedione | | | | | |
| Diethylhexyl butamidotriazone | | 2.00 | | 2.00 | 1.00 |
| Ethylhexyl triazone | 4.00 | | 3.00 | 4.00 | |
| 4-Methylbenzylidenecamphor | 4.00 | 4.00 | | | 2.00 |
| Octocrylene | | 4.00 | 4.00 | | 2.50 |
| Phenylene-1,4-bis(monosodium, | 1.00 | | 0.50 | | 2.00 |
| 2-benzimidazyl-5,7-disulfonic acid) | | | | | |
| Phenylbenzimidazole sulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Dioic acid | 0.3 | 0.10 | 0.50 | 1.00 | 0.20 |
| Tyrosine sulfate | 0.10 | 0.30 | 1.00 | 0.70 | 0.50 |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

39. Gel cream with tyrosine sulfate:

| | |
|---|---|
| Acrylate/C10-30 alkyl acrylate crosspolymer | 0.40 |
| Polyacrylic acid | 0.20 |
| Xanthan gum | 0.10 |
| Cetearyl alcohol | 3.00 |
| C12-15 alkyl benzoate | 4.00 |
| Caprylic/capric triglyceride | 3.00 |
| Cyclic dimethylpolysiloxane | 5.00 |
| Dimethicone polydimethylsiloxane | 1.00 |
| Dioic acid | 1.00 |
| Tyrosine sulfate | 0.50 |
| Glycerol | 3.00 |
| Sodium hydroxide | q.s. |
| Preservative | q.s. |
| Perfume | q.s. |
| Water | ad 100.0 |
| pH adjusted to 6.0 | |

40. W/O cream with tyrosine sulfate

| | |
|---|---|
| Polyglyceryl 3-diisostearates | 3.50 |
| Glycerol | 3.00 |
| Polyglyceryl 2-dipolyhydroxystearates | 3.50 |
| Dioic acid | 0.70 |
| Tyrosine sulfate | 0.70 |
| Preservative | q.s. |
| Perfume | q.s. |
| Water | ad 100.0 |
| Magnesium sulfate | 0.6 |
| Isopropyl stearate | 2.0 |
| Caprylyl ether | 8.0 |
| Cetearyl isononanoate | 6.0 |

41. W/O/W cream with tyrosine sulfate:

| | |
|---|---|
| Glyceryl stearate | 3.00 |
| PEG 100 stearate | 0.75 |
| Behenyl alcohol | 2.00 |
| Caprylic/capric triglyceride | 8.0 |
| Octyldodecanol | 5.00 |
| C12-15 alkyl benzoate | 3.00 |
| Dioic acid | 1.00 |
| Tyrosine sulfate | 1.00 |
| Magnesium sulfate (MgSO₄) | 0.80 |
| Ethylenediaminetetraacetic acid | 0.10 |
| Preservative | q.s. |
| Perfume | q.s. |
| Water | ad 100.0 |
| pH adjusted to 6.0 | |

42. Conditioner shampoo with pearly
luster and with tyrosine sulfate

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Pearlescent agent | 2.0 | 2.0 | 2.0 |
| Tyrosine sulfate | 0.05 | 0.1 | 0.01 |
| Dioic acid | 0.02 | 0.05 | 0.01 |
| Disodium EDTA | 0.1 | 0.2 | 0.15 |
| Preservative, perfume, thickener, | q.s. | q.s. | q.s. |
| pH adjustment and solubilizer | | | |
| Water, deionized | ad 100.0 | ad 100.0 | ad 100.0 |

The pH is adjusted to 6.

43. Clear conditioner shampoo with tyrosine sulfate

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Tyrosine sulfate | 0.01 | 0.08 | 0.05 |
| Dioic acid | 0.04 | 0.10 | 0.07 |
| Iminodisuccinic acid, Na salt | 0.2 | 0.3 | 0.8 |
| Preservative, perfume, thickener, | q.s. | q.s. | q.s. |
| pH adjustment and solubilizer | | | |
| Water, deionized | ad 100.0 | ad 100.0 | ad 100.0 |

The pH is adjusted to 6.

44. Clear light shampoo with volume
effect and with tyrosine sulfate

| | 1 | 2 | 3 |
|---|---|---|---|
| Sodium laureth sulfate | 10.0 | 10.0 | 10.0 |
| Cocoamidopropylbetaine | 2.5 | 2.5 | 2.5 |
| Tyrosine sulfate | 0.3 | 0.4 | 0.6 |
| Dioic acid | 0.05 | 0.1 | 0.01 |
| Disodium EDTA | 0.2 | 0.15 | 0.7 |
| Preservative, perfume, thickener, | q.s. | q.s. | q.s. |
| pH adjustment and solubilizer | | | |
| Water, deionized | ad 100.0 | ad 100.0 | ad 100.0 |

The pH is adjusted to 5.5.

1. A cosmetic or dermatological preparation comprising an active ingredient combination of at least one antioxidant or a derivative thereof and 8-hexadecene-1,16-dicarboxylic acid.

2. A preparation as claimed in claim 1, wherein the at least one antioxidant is selected from the group consisting of imidazoles, peptides, carotenoids, α-lipoic acid, lipoamide, aurothioglucose, propylthiouracil and other thiols, metal chelators, humic acid, bile acid, bile extracts, bilirubin, biliverdin, unsaturated fatty acids, folic acid, flavenoids, tocopherols, rutinic acid, ferulic acid, butylhydroxytoluene, butylhydroxyanisole, nordihydroguaiaretic acid, nordihydroguaiaretic acid, trihydroxybutyrophenone, kojic acid, uric acid, mannose, zinc and salts thereof, selenium compounds and enzymatic antioxidants.

3. A preparation as claimed in claim 1, wherein the at least one antioxidant is selected from the group consisting of urocanic acid, phytoene, lipoic acid, lipoamide, ferritin, desferal, billirubin, billiverdin, melanins, ubiquinone, ubiquinol, vitamin C and derivatives thereof, ascorbyl palmitate, magnesium ascorbyl phosphate, ascorbyl acetate, tocopherols and derivatives thereof, uric acid, α-glucosylrutin, calalase and superoxide dismutase.

4. A preparation as claimed in claim 1, wherein the at least one antioxidant is selected from the group consisting of urocanic acid, lipoic acid, lipoamide, melanins, ubiquinone, α-tocopherol, uric acid and catalase.

5. A preparation as claimed in claim 1, wherein the at least one antioxidant is present in a concentration of 0.001 to 30% by weight, based on the total weight of the preparation, and does not include vitamin E or derivatives thereof.

6. A preparation as claimed in claim 1, wherein the at least one antioxidant is present in a concentration of 0.05 to 20% by weight, based on the total weight of the preparation, and does not include vitamin E or derivatives thereof.

7. A preparation as claimed in claim 1, wherein the at least one antioxidant is present in a concentration of 0.1 to 10% by weight, based on the total weight of the preparation, and does not include vitamin E or derivatives thereof.

8. A preparation as claimed in claim 1, wherein the at least one antioxidant is present in a concentration of 0.001 to 10% by weight, based on the total weight of the preparation, and includes vitamin E or derivatives thereof.

9. A preparation as claimed in claim 1, wherein the 8-hexadecene-1,16-dicarboxylic acid is present in a concentration of 0.001 to 10% by weight, based on the total weight of the preparation.

10. A preparation as claimed in claim 1, wherein the 8-hexadecene-1,16-dicarboxylic acid is present in a concentration of 0.005 to 8% by weight, based on the total weight of the preparation.

11. A preparation as claimed in claim 1, wherein the 8-hexadecene-1,16-dicarboxylic acid is present in a concentration of 0.05 to 5% by weight, based on the total weight of the preparation.

12. A cosmetic or dermatological preparation comprising an active ingredient combination of α-lipoic acid and 8-hexadecene-1,16-dicarboxylic acid.

13. The preparation as claimed in claim 12, comprising a plant or animal extract comprising α-lipoic acid.

14. A preparation as claimed in claim 12, wherein the α-lipoic acid is present in a concentration of 0.001 to 10% by weight, based on the total weight of the preparation.

15. A preparation as claimed in claim 12, wherein the α-lipoic acid is present in a concentration of 0.01 to 5% by weight, based on the total weight of the preparation.

16. A preparation as claimed in claim 12, wherein the α-lipoic acid is present in a concentration of 0.1 to 2.0% by weight, based on the total weight of the preparation.

17. A preparation as claimed in claim 12, wherein the 8-hexadecene-1,16-dicarboxylic acid is present in a concentration of 0.001 to 10% by weight, based on the total weight of the preparation.

18. A preparation as claimed in claim 12, wherein the 8-hexadecene-1,16-dicarboxylic acid is present in a concentration of 0.005 to 8% by weight, based on the total weight of the preparation.

19. A preparation as claimed in claim 12, wherein the 8-hexadecene-1,16-dicarboxylic acid is present in a concentration of 0.05 to 5% by weight, based on the total weight of the preparation.

20. A cosmetic or dermatological preparation comprising an active ingredient combination of carnosine and 8-hexadecene-1,16-dicarboxylic acid.

21. A preparation as claimed in claim 20, comprising a plant or animal extract comprising carnosine.

22. A preparation as claimed in claim 20, wherein the carnosine is L-carnosine.

23. A preparation as claimed in claim 20, wherein the carnosine is present in a concentration of 0.001 to 10% by weight, based on the total weight of the preparation.

24. A preparation as claimed in claim 20, wherein the carnosine is present in a concentration of 0.01 to 1% by weight, based on the total weight of the preparation.

25. A preparation as claimed in claim 20, wherein the 8-hexadecene-1,16-dicarboxylic acid is present in a concentration of 0.001 to 10% by weight, based on the total weight of the preparation.

26. A preparation as claimed in claim 20, wherein the 8-hexadecene-1,16-dicarboxylic acid is present in a concentration of 0.005 to 8% by weight, based on the total weight of the preparation.

27. A preparation as claimed in claim 20, wherein the 8-hexadecene-1,16-dicarboxylic acid is present in a concentration of 0.05 to 5% by weight, based on the total weight of the preparation.

28. A cosmetic or dermatological preparation comprising an active ingredient combination of at least one bioquinone and 8-hexadecene-1,16-dicarboxylic acid.

29. A preparation as claimed in claim 28, comprising a plant or animal extract comprising at least one bioquinone.

30. A preparation as claimed in claim 28, wherein the at least one bioquinone includes coenzyme Q10.

31. A preparation as claimed in claim 30, wherein the coenzyme Q10 is present in a concentration of 0.000001 to 5% by weight, based on the total weight of the preparation.

32. A preparation as claimed in claim 30, wherein the coenzyme Q10 is present in a concentration of 0.01 to 1% by weight, based on the total weight of the preparation.

33. A preparation as claimed in claim 30, wherein the 8-hexadecene-1,16-dicarboxylic acid is present in a concentration of 0.001 to 10% by weight, based on the total weight of the preparation.

34. A preparation as claimed in claim 30, wherein the 8-hexadecene-1,16-dicarboxylic acid is present in a concentration of 0.005 to 8% by weight, based on the total weight of the preparation.

35. A preparation as claimed in claim 30, wherein the 8-hexadecene-1,16-dicarboxylic acid is present in a concentration of 0.05 to 5% by weight, based on the total weight of the preparation.

36. A cosmetic or dermatological preparation comprising at least one active ingredient selected from the group consisting of 3-[4-hydroxyphenylsulfate ester]-2-aminopropionic acid, 3-[3-hydroxyphenylsulfate ester]-2-aminopropionic acid (tyrosine O-sulfate ester), sulfonic esters thereof, phosphate esters thereof, and derivatives thereof, in combination with 8-hexadecene-1,16-dicarboxylic acid.

37. A preparation as claimed in claim 36, comprising a plant or animal extract comprising the at least one active ingredient.

38. A preparation as claimed in claim 36, wherein the at least one active ingredient is selected from the group consisting of 3-[3-hydroxyphenylsulfate ester]-2-aminopropionic acid (tyrosine O-sulfate ester), sulfonic esters thereof, phosphate esters thereof, and derivatives thereof,.

39. A preparation as claimed in claim 36, comprising effective amounts of said at least one active ingredient and of said 8-hexadecene-1,16-dicarboxylic acid.

40. A preparation as claimed in claim 39, wherein the at least one active ingredient is present in a concentration of 0.001 to 10% by weight, based on the total weight of the preparation.

41. A preparation as claimed in claim 39, wherein the at least one active ingredient is present in a concentration of 0.01 to 5% by weight, based on the total weight of the preparation.

42. A preparation as claimed in claim 39, wherein the at least one active ingredient is present in a concentration of 0.1 to 2.0% by weight, based on the total weight of the preparation.

43. A preparation as claimed in claim 39, wherein the 8-hexadecene-1,16-dicarboxylic acid is present in a concentration of 0.001 to 10% by weight, based on the total weight of the preparation.

44. A preparation as claimed in claim 39, wherein the 8-hexadecene-1,16-dicarboxylic acid is present in a concentration of 0.005 to 8% by weight, based on the total weight of the preparation.

45. A preparation as claimed in claim 39, wherein the 8-hexadecene-1,16-dicarboxylic acid is present in a concentration of 0.05 to 5% by weight, based on the total weight of the preparation.

46. A method for treating unwanted pigmentation of the skin or pigmentation disorders, comprising applying to the skin a cosmetic or dermatological preparation comprising an active ingredient combination of at least one antioxidant or a derivative thereof and 8-hexadecene-1,16-dicarboxylic acid.

47. A method as claimed in claim 46, wherein the at least one antioxidant includes α-lipoic acid.

48. A method as claimed in claim 46, wherein the at least one antioxidant includes carnosine.

49. A method as claimed in claim 46, wherein the at least one antioxidant includes at least one bioquinone.

50. A method as claimed in claim 46, wherein the at least one antioxidant includes at least one active ingredient selected from the group consisting of 3-[4-hydroxyphenylsulfate ester]-2-aminopropionic acid, 3-[3-hydroxyphenylsulfate ester]-2-aminopropionic acid (tyrosine O-sulfate ester), sulfonic esters thereof, phosphate esters thereof, and derivatives thereof.

51. A method for treating unwanted pigmentation of the hair or for lightening the hair, comprising applying to the hair a cosmetic or dermatological preparation comprising an active ingredient combination of at least one antioxidant or a derivative thereof and 8-hexadecene-1,16-dicarboxylic acid.

52. A method as claimed in claim 51, wherein the at least one antioxidant includes α-lipoic acid.

53. A method as claimed in claim 51, wherein the at least one antioxidant includes carnosine.

54. A method as claimed in claim 51, wherein the at least one antioxidant includes at least one bioquinone.

55. A method as claimed in claim 51, wherein the at least one antioxidant includes at least one active ingredient selected from the group consisting of 3-[4-hydroxyphenylsulfate ester]-2-aminopropionic acid, 3-[3-hydroxyphenylsulfate ester]-2-aminopropionic acid (tyrosine O-sulfate ester), sulfonic esters thereof, phosphate esters thereof, and derivatives thereof.
